(19) United States
(12) Patent Application Publication
(10) Pub. No.: US 2026/0033999 A1
(43) Pub. Date: Feb. 5, 2026

(54) TOPICAL APPLICATION FOR ABSORBENT ARTICLE

(71) Applicant: The Procter & Gamble Company, Cincinnati, OH (US)

(72) Inventors: Jose Enrique BETANCOURT, West Chester, OH (US); Kasey Marie HUDSON, Cincinnati, OH (US); Fang LIU, Mason, OH (US)

(21) Appl. No.: 19/284,902

(22) Filed: Jul. 30, 2025

Related U.S. Application Data
(60) Provisional application No. 63/678,137, filed on Aug. 1, 2024.

(51) Int. Cl. A61F 13/511; A61F 13/51

(52) U.S. Cl. CPC A61F 13/51113; A61F 2013/51004; A61F 2013/51028; A61F 2013/51117; A61F 2013/51169

(57) ABSTRACT

A feminine hygiene pad, comprising a topsheet lotion composition in discrete drops on individual fibers of the topsheet; wherein the discrete drops are on both the outer surface and between the outer surface and inner surface of the topsheet; wherein each drop is at most about 100 microns; and wherein the lotion composition has a viscosity from about 3.0 cP to about 5.0 cP at 80° C.

### CROSS REFERENCE TO RELATED APPLICATION

This application claims the benefit of U.S. Provisional Application No. 63/678,137, filed Aug. 1, 2024, which is incorporated by reference herein in its entirety.

### BACKGROUND

Absorbent articles of various designs have been used for many years for the purpose of intercepting, capturing, containing, and absorbing bodily exudates including menstrual fluid, urine, and feces, for the purpose of managing the exudate to avoid soiling of underwear, outer clothing, bedding, etc.

One type of absorbent article, the feminine hygiene pad (also known as a “sanitary napkin,” “menstrual pad,” etc.) has been used by women to manage discharge of menstrual fluid during menses. A typical feminine hygiene pad includes a liquid permeable topsheet which forms the body/wearing-facing surface of the pad; a liquid impermeable backsheet which forms the outward-facing surface of the pad and serves as a barrier to prevent fluid absorbed by the pad from migrating to and exiting the outward-facing surface of the pad; and an absorbent structure which serves fluid handling functions in any desired combination of lesser or greater extents, including fluid interception, distribution within the pad, absorption, containment, and storage, during the desired duration of wear/use of the pad. The typical pad is configured, shaped, and sized to be placed and worn inside the intended user's underpants in the crotch region thereof. Many pads include one or more deposits of adhesive on the outward-facing surface of the backsheet, to enable the wearer to affix the pad to the inside surface of the underwear to help hold it in a suitable position during wear/use.

Regarding the topsheet, generally it is desired by users of a feminine hygiene pad that the topsheet readily admit menstrual fluid and facilitate its rapid passage in a z-direction downward towards underlying absorbent components (rapid acquisition) while resisting x-y direction spreading of, and staining by, menstrual fluid. Ideally, the topsheet further resists reacquiring fluid from underlying absorbent components, allowing it to move in a z-direction upwardly therethrough (rewetting) back to the wearer-facing surface, causing a wet feel for the wearer and/or a perception of incomplete absorption and less effective protection. And it is of course desired that the topsheet feels soft and comfortable against the wearer's skin, and dry, including after a fluid insult.

A variety of materials have been developed and used to form topsheets for feminine hygiene pads. In some examples, a topsheet may be formed of a polymeric film having a pattern of apertures formed therein. The apertures serve to allow discharged fluid to pass through the topsheet to absorbent components of the pad disposed therebeneath. Some segments of the consumer market, however, may not prefer film topsheets for various reasons, which may include their feel against the skin.

In other examples, topsheets can be formed of a fibrous nonwoven web material. Myriad combinations of features of a nonwoven are possible. While the nonwoven materials can be perceived as softer, they can sometimes retain the body fluids and thus not be perceived as clean. Therefore, nonwoven topsheets can have the drawback of trapping the menses in the topsheet.

To address this, absorbent articles may use a more open nonwoven topsheet structure that can reduce capillarity that can “trap” menses between fibers. But there remains a need to better move fluid in the z-direction to reach the absorbent core that retains the fluid. The present invention provides a topsheet lotion that minimizes fluid left in the topsheet.

### SUMMARY OF THE INVENTION

A feminine hygiene pad, comprising:
a liquid permeable topsheet having an inner surface oriented toward the interior of said pad and an outer surface oriented toward the skin of a wearer when said pad is being worn, said topsheet comprising individual nonwoven fibers;
a backsheet;
an absorbent core disposed between the topsheet and the backsheet; and
a topsheet lotion composition;
wherein the topsheet lotion composition comprises discrete drops on individual fibers of the topsheet;
wherein the discrete drops are on both the outer surface and between the outer surface and inner surface of the topsheet;
wherein each drop is at most about 100 microns; and
wherein the lotion composition has a viscosity from about 3.0 cP to about 5.0 cP at 80° C.

### BRIEF DESCRIPTION OF THE DRAWINGS

While the specification concludes with claims particularly pointing out and distinctly claiming the subject matter that is regarded as the present invention, it is believed that the invention will be more fully understood from the following description taken in conjunction with the accompanying drawings. Some of the figures may have been simplified by the omission of selected elements for the purpose of more clearly showing other elements. Such omissions of elements in some figures are not necessarily indicative of the presence or absence of particular elements in any of the exemplary embodiments, except as may be explicitly delineated in the corresponding written description. None of the drawings are necessarily to scale.

FIG. 1A is a schematic plan view (along a z-direction) depiction of an example of an absorbent article.

FIG. 1B is a schematic plan view (along a z-direction) depiction of examples of absorbent core component layers of the absorbent article depicted in FIG. 1A.

FIG. 2 is a plan view (along a z-direction) image of a portion of a nonwoven web material having a pattern of apertures therethrough.

FIG. 3 is a plan view (along a z-direction) magnified image of a portion of a nonwoven web material having an aperture therethrough.

FIG. 4A is a magnified image of lotion applied to the topsheet.

FIG. 4B is a portion of a nonwoven web material having drops from the lotion.

FIG. 5 is a top view of a strikethrough plate used in the Acquisition Time and Rewet Measurement Method described herein.

FIG. 6 is a bottom view of the strikethrough plate used in the Acquisition Time and Rewet Measurement Method described herein.

FIG. 7A is a cross section view of the strikethrough plate used in the Acquisition Time and Rewet Measurement Method described herein, taken along a plane defined by the z-direction and line A-A shown in FIG. 5.

FIG. 7B is a cross section view of the strikethrough plate used in the Acquisition Time and Rewet Measurement Method described herein, taken along a plane defined by the z-direction and line B-B shown in FIG. 5.

FIG. 8 is a chart presenting second acquisition times measured for samples 1-8 of prototype feminine hygiene pads (P1-P8), measured using the Acquisition Time and Rewet Measurement Method described herein).

FIG. 9 is a chart presenting sums of surface free fluid (SFF) and rewet measured for samples 1-8 of prototype feminine hygiene pads (P1-P8) with different Formulas (structuring agent) and amounts (gsm), measured using the Acquisition Time and Rewet Measurement Method described herein.

### DETAILED DESCRIPTION OF THE INVENTION

### Definitions

As used herein, the following terms shall have the meaning specified thereafter:

“Absorbent article” refers to wearable devices, which absorb and/or contain liquid, and more specifically, refers to devices, which are placed against or in proximity to the body of the wearer to absorb and contain the various exudates discharged from the body. Absorbent articles can include diapers, training pants, adult incontinence undergarments (e.g., liners, pads, and briefs) and/or feminine hygiene products, including feminine hygiene pads (also known as, for example, “sanitary napkins”, “menstrual pads”, “panty liners”, etc.).

The term “integrated” as used herein is used to describe fibers of a nonwoven material which have been intertwined, entangled, and/or pushed/pulled in a positive and/or negative Z-direction (direction of the thickness of the nonwoven material). Some exemplary processes for integrating fibers of a nonwoven web include spunlacing and needlepunching. Spunlacing (also known as “hydroentangling” or (“hydroenhancing”) uses a plurality of high pressure water jets directed at a precursor batt or accumulation of fibers being conveyed along a machine direction, to entangle the fibers. Needlepunching (also known as “needling”) involves the use of specially-featured needles to mechanically push and/or pull fibers, of a precursor batt or accumulation of fibers, in a z-direction, to entangle them with other fibers in the batt or accumulation.

The term “carded” as used herein is used to describe structural features of the fluid management layers described herein. A carded nonwoven web is formed of fibers which are cut to a specific finite length, otherwise known as “staple length fibers.” Staple length fibers may be of any selected length. For example, staple length fibers may be cut to a length of up to 120 mm, to a length as short as 10 mm. However, if fibers of a particular group are staple length fibers, then the length of each of the fibers in the carded nonwoven is approximately the same, i.e., the staple length. Where fibers of more than one composition are included in a nonwoven web, for example, a web including polypropylene fibers and viscose fibers, the length of each fiber of the same composition may be substantially the same, while the respective staple fiber lengths of the respective fiber compositions may differ.

In contrast to staple fibers, filaments such as those produced by spinning, e.g., in a spunbond or meltblown nonwoven web manufacturing processes, are not ordinarily staple length fibers. Instead, these filaments are sometimes characterized as “continuous” fibers, meaning that they are of a relatively long and indeterminate length, not cut to a specific length following spinning, as their staple fiber counterparts are.

“Lateral”, with respect to an absorbent article such as a feminine hygiene pad, or a component thereof, refers to a direction parallel to a horizontal line tangent to the front surfaces of the upper portions of wearer's legs proximate the torso, when the pad is being worn normally and the wearer has assumed an even, square, normal standing position. A “width” dimension of any component or feature of an article such as a feminine hygiene pad is measured along the lateral direction. When the article or component thereof is laid out flat on a horizontal surface, the “lateral” direction corresponds with the lateral direction relative to the structure when it is worn, as defined above. With respect to an article such as a feminine hygiene pad that is opened and laid out flat on a horizontal planar surface, “lateral” refers to a direction perpendicular to the longitudinal direction and parallel to the horizontal planar surface. With respect to a feminine hygiene pad, the term “y-direction” is interchangeable with the term “longitudinal direction.”

The “lateral axis” of an absorbent article such as a feminine hygiene pad or component thereof is a lateral line lying in an x-y plane and equally dividing the length of the pad or the component when it is laid out flat on a horizontal surface. A lateral axis is perpendicular to a longitudinal axis.

“Longitudinal”—with respect to an absorbent article such as a feminine hygiene pad, or a component thereof, refers to a direction perpendicular to the lateral direction. A “length” dimension of any component or feature of the article is measured along the longitudinal direction from its forward extent to its rearward extent. When an article such as a feminine hygiene pad or component thereof is laid out flat on a horizontal surface, the “longitudinal” direction is perpendicular to the lateral direction relative the pad when it is worn, as defined above. With respect to a feminine hygiene pad, the term “y-direction” is interchangeable with the term “longitudinal direction.”

The “longitudinal axis” of a feminine hygiene pad or component thereof is a longitudinal line lying in an x-y plane and equally dividing the width of the pad or component when the pad is laid out flat on a horizontal surface. A longitudinal axis is perpendicular to a lateral axis.

Herein, the term “rayon” is used generically to include any fiber spun from regenerated cellulose, including but not limited to viscose, lyocell, etc.

“x-y plane,” with reference to an absorbent article, such as a feminine hygiene pad, or component thereof, when laid out flat on a horizontal surface, means any horizontal plane occupied by the horizontal surface or any layer of the article or component. With respect to manufacture or processing of a material web, the term “x-y plane” refers to a plane substantially occupied by a major surface of the material web.

With respect to manufacture or processing of a material web, the term “x-direction” is interchangeable with the term “cross direction.”

With respect to manufacture or processing of a material web, the term “y-direction” is interchangeable with the term “machine direction.”

“z-direction,” with respect to an absorbent article, such as a feminine hygiene pad or component thereof, when laid out flat on a horizontal surface, is a direction perpendicular/orthogonal to the x-y plane. With respect to manufacture or processing of a material web, the term “z-direction” refers to a direction orthogonal to an x-y plane substantially occupied by a major surface of the material web.

The terms “top,” “bottom,” “upper,” “lower,” “over,” “under,” “beneath,” “superadjacent,” “subjacent,” and similar terms relating to relative vertical positioning, when used herein to refer to layers, components or other features of an absorbent article such as a feminine hygiene pad, are relative the z-direction and are to be interpreted with respect to the pad as it would appear when laid out flat on a horizontal surface, with its wearer-facing surface oriented upward and outward-facing surface oriented downward.

With respect to an absorbent article such as a feminine hygiene pad, or a component or structure thereof, “wearer-facing” is a relative locational term referring to a feature of the component or structure that when in use that lies closer to the wearer than another feature of the component or structure. For example, a topsheet has a wearer-facing surface that lies closer to the wearer than the opposite, outward-facing surface of the topsheet.

With respect to an absorbent article such as a feminine hygiene pad, or a component or structure thereof, “outward-facing” is a relative locational term referring to a feature of the component or structure that when in use that lies farther from the wearer than another feature of the component or structure. For example, a topsheet has an outward-facing surface that lies farther from the wearer than the opposite, wearer-facing surface of the topsheet.

“Machine Direction” or “MD” as used herein with respect to an absorbent article such as a feminine hygiene pad or component thereof, refers to a direction parallel to the flow of the article or component through processing/manufacturing equipment.

“Cross Direction” or “CD” as used herein with respect to an absorbent article such as a feminine hygiene pad or component thereof, refers to a direction perpendicular/orthogonal to the machine direction.

“Predominant,” and forms thereof, when used to characterize a quantity of weight, volume, surface area, etc., of an absorbent article or component thereof, constituted by a composition, material, feature, etc., means that a majority of such weight, volume, surface area, etc., of the absorbent article or component thereof is constituted by the composition, material, feature, etc.

“Plant-derived” refers to ingredients of a composition that are directly obtained from plants or are derived from one or more processing operations applied to plants or portions thereof. These processing operations may include, but are not limited to, purification, heating, fractionization, hydrolysis, esterification, condensation, fermentation, distillation, extraction, and maceration.

“Emollient” refers a material that protects against wetness or irritation and softens, soothes, supples, coats, lubricates, moisturizes, protects and/or cleanses the skin.

### General—Absorbent Article; Feminine Hygiene Pad

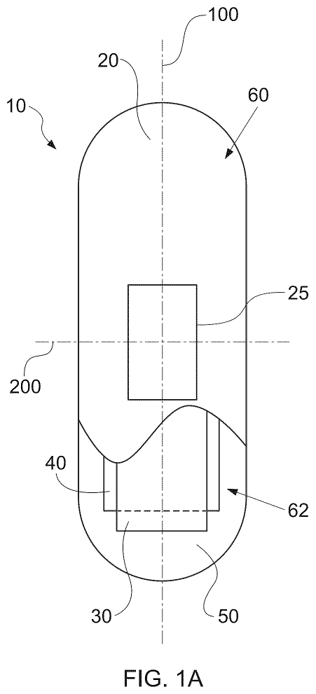

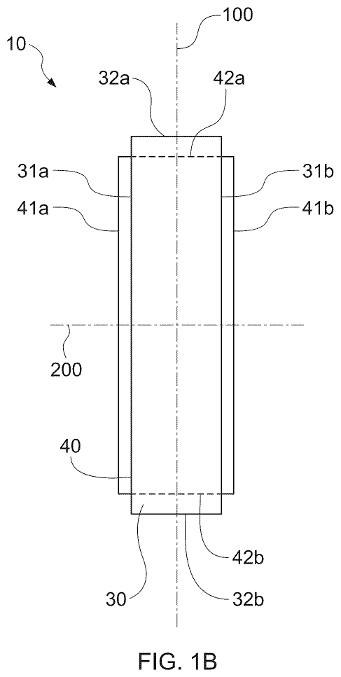

Referring to FIG. 1A, an absorbent article as contemplated herein, such as a feminine hygiene pad 10, will include a wearer-facing surface and an opposing outward-facing surface. A liquid permeable topsheet 20 may form at least a portion of the wearer-facing surface and a liquid impermeable backsheet may form at least a portion of the outward-facing surface. An absorbent core including an absorbent structure 40 is disposed between the topsheet and the backsheet, and a fluid management layer 30 may be included and disposed between the absorbent structure 40 and the topsheet 20. (A fluid management layer as described herein is sometimes known in the art as an “acquisition/distribution layer” “distribution layer” or “secondary topsheet”, whose purpose is to dissipate energy from a fluid gush to the extent needed, provide a temporary volume of space for discharged fluid to occupy during the time required for an underlying absorbent structure to imbibe and absorb the fluid, and to distribute the fluid across the absorbent structure to maximize effective use thereof.) Non-limiting examples of absorbent articles sharing these features include feminine hygiene pads (also known as “sanitary napkins”, “menstrual pads,” etc.), disposable incontinence pads, disposable incontinence underwear, disposable baby diapers and disposable baby/child training pants.

The topsheet 20 and the backsheet 50 may be joined together to form and define an outer periphery of the pad 10. The absorbent structure 40 and the fluid management layer 30 will each be sized to have outer perimeters disposed laterally and longitudinally inboard of the outer periphery. The absorbent structure 40 and the fluid management layer 30 may be dimensioned and shaped substantially similarly or identically to each other in the x-y directions, or they may have respective differing x-y dimensions and/or shapes. One or both may be manufactured to have a rectangular shape as suggested in FIG. 1A, or one or both may be manufactured to have any other suitable shape, such as an oval shape, stadium shape, rounded rectangle shape, hourglass shape, peanut shape, etc. Shapes having concave profiles along the longitudinal edges may in some examples provide for enhanced comfort and/or conformity with the wearer's body.

The topsheet 20 may be joined to the backsheet 50 by attachment any suitable attachment mechanism. The topsheet 20 and the backsheet 50 may be joined directly to each other in the article periphery and may be indirectly joined together by directly joining them to the absorbent structure 40, the fluid management layer 30, and/or additional layers disposed between the topsheet 20 and the backsheet 50. This indirect or direct joining may be accomplished by any suitable attachment mechanism known in the art. Non-limiting examples of attachment mechanisms may include e.g., fusion bonds, ultrasonic bonds, pressure bonds, adhesive bonds, or any suitable combinations thereof.

### Topsheet

### General

Generally, it is desirable that the topsheet 20 be compliant, soft feeling, and non-irritating to the wearer's skin. Suitable topsheet materials include a liquid pervious material that is oriented towards and contacts the body of the wearer permitting bodily discharges to rapidly penetrate through it without allowing fluid to flow back through the topsheet to the skin of the wearer. The topsheet, while being capable of allowing rapid transfer of fluid through it, may also provide for the transfer or migration of a lotion composition therefrom to facing surfaces of a wearer's skin. The topsheet may be formed of or include a nonwoven material.

Nonwoven fibrous topsheets 20 may be produced by any known procedure for making nonwoven webs, nonlimiting examples of which include spunbond processes, carding, wet-laying, air-laying, meltblowing processes, needle-punching, mechanical entangling, thermo-mechanical entangling, and hydroentangling.

Nonwoven materials suitable for use as a topsheet may include one strata of fibers or may be laminate of multiple nonwoven strata, which may comprise the same or different compositions (e.g., spunbond-meltblown laminate). In one specific example, the topsheet is a carded, air-through bonded nonwoven.

Topsheets contemplated herein do not include any predominant fraction of topsheet x-y surface area occupied by film. Some currently known topsheets for feminine hygiene pads include an apertured film, such as a hydroformed film or vacuum-formed film, alone or in combination with an adjacently-disposed nonwoven web material. The film may help to prevent liquids from resurfacing and contacting the wearer. The inventors have found, however, that a topsheet having the features described herein, particularly in combination with the fluid management layer described herein and the lotion composition, can effectively prevent rewet to a comparable degree or better, than pads having topsheets comprising film across a predominant portion of topsheet x-y surface area. Without being bound by theory, it is believed that the careful selection of the fiber types and lotion treatment in each of the strata in the fluid management layer, and the linear densities of the fiber types, can result in a desired combination of suitably low fluid acquisition time, and low rewet, overcoming the typical tradeoff in these conflicting objectives associated with prior nonwoven topsheets. The improved performance is evident from the combination of the unique nonwoven topsheet with lotion treatment of the present disclosure.

### Basis Weight

In some examples, a nonwoven topsheet material as contemplated herein may be manufactured to have a basis weight of about 15 gsm to 80 gsm, more preferably about 20 gsm to 60 gsm, or most preferably about 20 gsm to 40 gsm, specifically reciting all values within these ranges and any ranges created thereby. In particular examples, the topsheet nonwoven may be manufactured to a basis weight of about 18 gsm to 40 gsm, more preferably about 20 gsm to 30 gsm, even more preferably about 22 gsm to 26 gsm, specifically reciting all values within these ranges and any ranges created thereby. The range of desirable basis weight is influenced, at the lower end of the range, by the need for a level of web tensile strength needed for processing, and by consumer preferences for a level of opacity and substantiality of loft, feel and appearance. The range of desirable basis weight is influenced, at the upper end of the range, by the need for suitable rapid fluid acquisition and passage of fluid through the topsheet, and material cost concerns.

### Fiber Composition

Nonlimiting examples of woven and nonwoven materials suitable for use as the topsheet include fibrous materials made from natural fibers, e.g., cotton, including 100 percent organic cotton, modified natural fibers, semi-synthetic fibers (e.g., fibers spun from regenerated cellulose) synthetic fibers (e.g., fibers spun from polymer resin(s)), or combinations thereof. Synthetic fibers may include fibers spun from single polymers or blends of polymers.

However, in some examples it may be desired that nonwoven web of the topsheet include less than 10 percent, more preferably less than 5 percent, and even more preferably less than 1 percent by weight of any combination of cotton fibers, other plant fibers, rayon fibers or monocomponent fibers comprising polyester or polyamide. Such fibers are often hydrophilic in nature and thereby may tend to cause the topsheet to retain fluid rather than pass it along to absorbent components below. For the same reason, such fibers may tend to cause the topsheet to be prone to rewetting.

Synthetic fibers may include monocomponent fibers, bicomponent fibers or multicomponent fibers. (Herein, bi- or multicomponent fibers are fibers having cross sections divided into distinctly identifiable component sections, each formed of a single polymer or single homogeneous polymer blend, distinct from that of the other section(s). Such fibers and processes for making them are known in the art. Examples of bicomponent fiber configurations with substantially round cross sections include side-by-side or “pie slice” configurations, eccentric sheath-core configurations and concentric sheath-core configurations.

Nonwoven topsheets contemplated herein may include fibers having myriad combinations of constituent chemistries. For example, fibers may be spun from thermoplastic polymeric materials, such as polyethylene (PE) and/or polyethylene terephthalate (PET). Fibers may be spun in the form of bi-component fibers. In some examples, bi-component fibers may have a core component of a first polymer (for example, PET) in combination with another polymer as a sheath component, in a sheath-core bicomponent configuration. In more particular examples, PE may form the sheath component in combination with a PET core component. Fibers that include a PET component may be selected to help provide bulk and resilience and a resulting cushiony feel to the nonwoven web. Additionally, fibers that include a PET component, having resilience, help the web retain the area and dimensions of apertures created therethrough, if included.

Other polymeric materials may be included. For example, fibers spun of polypropylene, polyethylene, co-polyethylene terephthalate, co-polypropylene, and other thermoplastic resins may be included. It may be desired that the polymer with the lower melting temperature form the sheath component where sheath-core bi-component fibers are included. Additionally, without wishing to be bound by theory, it is believed that the use of polyethylene terephthalate as a core component can help impart resilience to the fiber, and as a result, to the topsheet.

Polyethylene, as a polymer component from which fibers may be spun, has a relatively lower melting temperature, and exhibits a relatively slick/silky surface feel as compared with other potentially useful thermoplastic polymers. PET has a relatively higher melting temperature and exhibits relatively greater stiffness and resiliency. Accordingly, in some examples topsheet nonwoven fibers that are of a sheath-core bicomponent configuration may be desired, in which the sheath component is predominantly polyethylene, and the core component is predominantly PET. The polyethylene is useful for imparting the fibers and thus the topsheet with a silky feel, and for enabling inter-fiber bonding via heat treatment that causes sheaths of adjacent/contacting fibers to melt and fuse at the lower melting temperature of the polyethylene, while the PET is useful for imparting resilience, and will not melt at lower temperatures that will melt PE in the heat treatment process.

### Surface Treatment (Hydrophilicity/Hydrophobicity)

Depending upon the chemical composition thereof, surfaces of fibers will be, inherently, cither hydrophilic or hydrophobic, to varying extents. For example, surfaces of fibers spun or otherwise formed from some types of polymers such as polyethylene and polypropylene will be, inherently, hydrophobic. In contrast, surfaces of other types of fibers, such as rayon fibers, will be inherently hydrophilic. Surfaces of natural fibers may be inherently hydrophilic or hydrophobic, but this may depend upon the processing the fibers have undergone. For example, cotton fibers as harvested bear coatings of natural oils and/or waxes and as such their surfaces are hydrophobic. After they have undergone processes including scouring and bleaching, however, the oils and/or waxes will have been stripped away, rendering the fiber surfaces hydrophilic.

Manufacturers and/or suppliers of spun synthetic staple fibers currently apply coatings, in the form of surface finishing agents or processing aids, to the fibers, for purposes of providing lubricity in, e.g., carding processes. These surface finishing agents or processing aids may be formulated to be either hydrophobic or hydrophilic, and to be substantially durable for purposes herein, in that they will not dissolve in aqueous fluids over the ordinary duration of wear of an absorbent article. Thus, a manufacturer or supplier of spun synthetic staple fibers may offer fibers with either hydrophobic or hydrophilic surface finishes, and currently, several manufacturers in the nonwovens materials industry do this.

Noting that spun synthetic staple fibers may be obtained with either inherently hydrophobic or hydrophilic surfaces, or obtained with surface finishes that render their surfaces hydrophilic or hydrophobic at the purchaser's option, it may be desirable to choose fibers with surfaces that are either hydrophilic (“hydrophilic fibers”) or hydrophobic (“hydrophobic fibers”), or choose a blend of fibers of both types.

In some examples it may be preferable that the fiber constituents of the topsheet be, by weight, predominantly, substantially, or entirely hydrophobic, or rendered hydrophobic via fiber surface finish. A topsheet formed of a nonwoven web with predominately hydrophobic fiber constituents will be resistive to rewetting. On the other hand, if the sizes of the pores or inter-fiber voids within the fibrous structure of such nonwoven web are not sufficiently large, the topsheet may resist the passage of fluid from the wearing facing surface through to the absorbent core components of the article therebeneath, i.e., will not readily/rapidly acquire fluid, unless other features are included in combination, as described herein.

In other examples, fibers constituting portions, a majority (by surface area), or all, of the section of web material from which of the topsheet is formed, may be a blend of both hydrophobic fibers and hydrophilic fibers. In such examples, the hydrophilic fibers can serve to help wick fluid from the wearer-facing surface of the topsheet down to the absorbent core components beneath, while the hydrophobic fibers can serve to help the topsheet resist rewetting.

Accordingly, in some examples the topsheet nonwoven may include a mix of hydrophobic and hydrophilic fibers, or be all hydrophobic or all hydrophilic. For example, the nonwoven may include at least about 40 percent, more preferably at least about 50 percent, or most preferably at least about 60 percent hydrophilic fibers by weight of the fibers, specifically including all values within these ranges and any ranges created thereby. In more particular examples, the nonwoven topsheet may comprise about 40 percent to 70 percent, more preferably about 45 percent to 68 percent, or most preferably from about 50 percent to 65 percent, by weight, hydrophilic fibers, specifically reciting all values within these ranges and any ranges created thereby. The topsheet nonwoven may include a blend of hydrophilic fibers and hydrophobic fibers in a weight ratio of hydrophilic fibers to hydrophobic fibers of 30:70 to 70:30, more preferably 35:65 to 65:35, and even more preferably 40:60 to 60:40. As noted above, the hydrophilicity of the hydrophilic fibers may be affected by application of a surface treatment composition.

Without wishing to be bound by theory, it is believed that where a majority of the fibers are hydrophilic, fluid acquisition speed can be improved by combination with other features described herein, while not overly impacting rewet in a negative or unacceptably negative manner. Where less rewet is the goal, then the converse may be true. In this circumstance, a higher weight fraction of hydrophobic fibers may be desired.

### Linear Density

Fibers are typically manufactured, selected, and purchased by linear density specification, such expressed as denier or decitex. For fibers of a given polymer constitution, linear density correlates with fiber size/diameter.

In some examples, the fibers constituting the topsheet may be selected to have an average linear density of about 1.0 to 3.0 denier, more preferably about 1.5 to 2.5 denier, and even more preferably about 1.8 to 2.2 denier, and all combinations of subranges within these ranges are contemplated herein. Fibers with varying linear densities within the ranges set forth above may be selected and included as well.

In other examples, the fibers constituting the topsheet may be selected to have an average linear density of about 3.0 to 5.0 denier, more preferably about 3.5 to 4.5 denier, and even more preferably about 3.8 to 4.2 denier, and all combinations of subranges within these ranges are contemplated herein. It has been learned that fibers selected within these ranges, in combination with other features disclosed herein, may be deemed to constitute a topsheet material of acceptable softness to many consumers, as well as to provide other advantages over smaller fibers.

One advantage is that the relatively larger fibers generally provide a nonwoven web material with relatively larger inter-fiber/intra-web spaces or voids therewithin, thereby providing larger passageways through which fluid may more rapidly travel through the nonwoven from the wearer-facing side through to the outward-facing side (and thus to absorbent components below the topsheet). Additionally, although relatively larger fibers of a given composition are stiffer than smaller fibers of similar composition, which may somewhat compromise surface “softness” attributes, the greater fiber stiffness can also enhance a feeling of greater resiliency, springy or cushiony feel to the topsheet nonwoven.

### Staple Fiber Length

Suitable fibers may be staple fibers having a length of at least about 30 mm, 40 mm, or 50 mm, up to about 55 mm, or about 30 to 55 mm, or about 35 to 52 mm, reciting for said range every 1 mm increment therein. In one particular example, staple fibers may have a length of about 38 mm.

### Apertures

The inventors have found that, in topsheet nonwovens that are formed of fibers of relatively small size/linear density and/or fibers that are predominantly, substantially or entirely hydrophobic, acquisition speed may be substantially increased by forming a pattern of apertures through the web in addition to the lotion composition. In examples in which fibers are of relatively larger size/linear density as also contemplated herein, forming and including a pattern of apertures may not be deemed necessary in some circumstances, but may be helpful in other circumstances, for the purpose of providing more surface-to-surface passageways of relatively greater size, enabling more rapid passage of discharged body exudates down through the topsheet.

Generally, the preferred apertures will have sizes that are substantially larger than the average pore/void size within the nonwoven web. The apertures may be formed by any suitable, known pin punching process. The process may include use of pins arranged in any desired pattern and radially extending from a pinned cylindrical roller, coupled with a mating cylindrical roller having pin receiving holes in its surface. One or both rollers may be heated to a temperature sufficient to cause softening and plastic deformation of the nonwoven web fibers, without melting them. Passage of the nonwoven web material through the nip between these rollers can effect enduring or substantially permanent displacement of the positions of the fibers along x- and y-directions, as well as the z-direction, within the nonwoven structure, thereby creating apertures through the web that substantially retain their sizes and shapes as the web is manipulated in later/downstream processes such rolling, unrolling and absorbent article manufacturing processes. Preferably, the aperture-forming process follows bonding of the web via heat treatment, to provide for more reliable formation of more dimension- and shape- stable apertures.

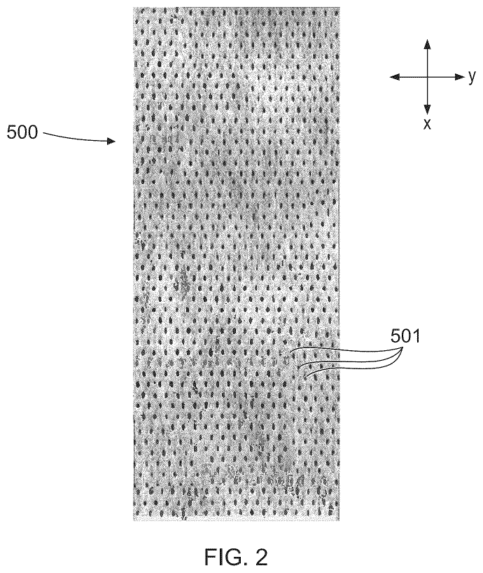

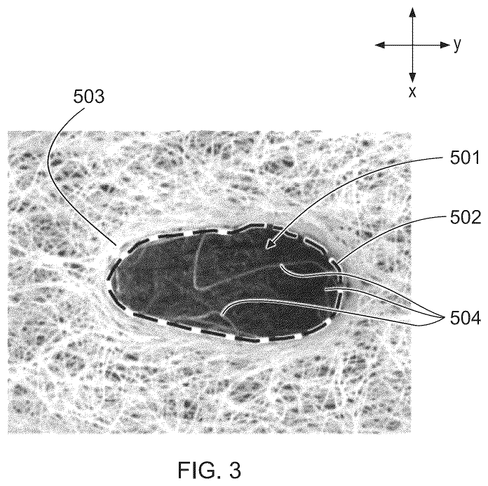

An example of a section of topsheet nonwoven web material 500 having a pattern of apertures 501 therethrough is depicted in FIG. 2. A magnified image of example of an aperture through a nonwoven web material is depicted in FIG. 3. Apertures are distinguishable from randomly-disposed inter-fiber/intra web spaces or voids through the nonwoven web material, in that they are created by readily discernible displacements of fibers, along x-y directions, resulting in concentrated groups of displaced fibers that define the perimeter of a z-direction opening through the nonwoven web that is relatively larger than the randomly disposes pores or voids between and among the fibers constituting the material. Apertures may be created through the web via a process and equipment configured to impart an average x-y dimension aperture area of 0.5 mm² to 2.5 mm², preferably about 0.6 mm² to 1.2 mm², and all combinations of subranges within these ranges are contemplated herein. Herein, the x-y dimension area of an aperture is defined by visually discernible inside edges of the concentrations of displaced fibers 503 about the perimeter of the aperture. Stray individual fibers that may have escaped the main structure and/or the concentrations of displaced fibers about the perimeter, and cross into or through the main open area of the aperture (by way of illustrative example, stray individual fibers 504 shown in FIG. 3) are not considered subtractive from the aperture area for purposes herein. Further, without wishing to be bound by theory, it is believed that the where the shapes of the apertures are too oblong or narrow, fluid acquisition speed may be negatively impacted. Accordingly, it may be desired that the apertures have a limited maximum average x-y direction aspect ratio (greatest dimension:smallest dimension in x-y directions). Thus, it may be desired that the average aspect ratio of the apertures be about 2.5:1 to 1:2.5, more preferably about 1.5:1 to 1:1.5, or most preferably about 1:1, and all combinations of subranges within these ranges are contemplated herein. Further, it is preferable for purposes of retaining structural integrity of the web and shape integrity of the apertures, that the x-y plane shapes of the majority or all of the apertures in, at least, the region of interest 25 (“ROI,” defined below; see FIG. 1A) if not the majority or entirety of the topsheet, be rounded shapes (e.g., circular, oval, ovoid, elliptical, stadium, etc.), having no sharp corners. Accordingly, it may be desired that the pinned roller used to create the apertures have pins that do not have sharp corners, when viewed along a radially inward direction toward the axis of the roller.

Collectively, the aperture areas of all the apertures in the portion of interest of the topsheet amount to an open x-y plane area (“open area”) in the topsheet nonwoven. In combination with a desired average aperture size, the inventors have identified a desired open area, in order to effectively mitigate potential obstacles to fluid acquisition that may result from constitution of fibers of finer denier and/or fibers that are predominantly hydrophobic. Accordingly, it may be desired that apertures, if included, collectively provide an open area of 6 percent to 25 percent, more preferably 8 percent to 18 percent, and even more preferably 10 percent to 15 percent, and all combinations of subranges within these ranges are contemplated herein. It is preferred that such amount of open area be present in substantially the entirety of the portion of the topsheet overlying the fluid management layer and/or absorbent structure, or at least, in the region of interest 25 (“ROI”) defined below (and see FIG. 1A). The lower limits of these ranges are imposed by the need for efficacy/performance; the apertures should provide at least a minimum amount of open area in order to be effective as may be included for the purposes described herein. The upper limits of these ranges are imposed by the need for consumer acceptance; if the open area is too great, consumers may perceive that the topsheet is fragile or of poor quality; and further, the topsheet becomes less effective at retaining fluid therebeneath, and at masking staining by absorbed fluid present in the absorbent components beneath the topsheet.

Referring to FIG. 1A, for purposes contemplated herein, a region of interest 25 (“ROI”) is a rectangular section of the topsheet that is 60.0 mm long in the longitudinal direction and 30.0 mm wide in the lateral direction, and is centered at the longitudinal and lateral center, in an x-y plane, of the fluid management layer. The percent fraction open area of the ROI 25 is the fraction of the x-y area therewithin that is open therethrough in the z-direction, by the collective presence of the apertures 501 therewithin. Expressed differently, the percent fraction open area within the ROI is the total x-y area of the apertures within the ROI, divided by 1,800 mm², times 100%.

The percent fraction open area in the ROI may be obtained in some examples from the specifications given to or provided by the manufacturer of the topsheet nonwoven web material. Where this is unavailable, it may be measured via any suitable measurement technique that may applied, in a manner consistent with the description of the x-y dimension area of an aperture area and description of “open area,” above, which may include but is not limited to the Apertures Open Arca Measurement Method set forth below.

### Bonding

Generally, it is desirable that the fibers forming the topsheet nonwoven be bonded following the carding/fiber laydown process, to impart a fabric-like structure and tensile strength (in both the MD and the CD) needed for the web to substantially retain its structure in downstream/later processes, and in the form of a topsheet, during use by a user/wearer. As an alternative to other methods of bonding such as mechanical compression spot bonding (with or without application of heating energy), adhesive bonding, etc., it has been found that bonding via air-through heating is effective for creating fiber-to-fiber bonds and imparting structure integrity to the web, while preserving inter-fiber pore/void size and loft, and imparting resiliency, to the nonwoven. In examples of suitable processes, air heated to the selected heating temperature is blown and/or drawn (via vacuum) through the carded fiber web as it is conveyed on a carrier belt along a machine direction, through an oven or heating chamber. When operating parameters including heating air temperature and velocity, and exposure time, are appropriately adjusted, a plurality of randomly distributed fiber-to-fiber bonds may be created within the fiber network, which impart structural integrity to the web. When constituent fibers are, for example, sheath-core bicomponent fibers in which the sheath component is a polymer having a melting temperature lower than that of the core component, the process may be configured such that fusion bonds form between sheaths of adjacent contacting fibers without complete melting and loss of structure of the sheaths, while the cores remain in place, un-melted. In such process, the bonds may be formed without application of compression, and thus, without associated loss of caliper of the web and reduction in size of the inter-fiber pores/voids.

### Topsheet Lotion

The absorbent article or feminine hygiene pad may include a topsheet lotion composition. The inventive topsheet lotion composition minimizes fluid left in the topsheet, that is, helps move menses from the wearer-facing surface of the topsheet down therethrough to the fluid management and/or absorbent structure layers beneath, allowing for a better clean and dry experience for the wearer, balanced with the soft cushioning of the nonwoven topsheet. The mechanism of the lotion composition to achieve the better fluid handling involves changing the surface tension of the interface of the menses and fibers and also lubricating some of the surface of the fibers. The lotion composition is a gentle topical material, a skin emollient like a balm. It may be comprised of at least about 99% plant derivatives and may be free of, or substantially free of water.

The topsheet lotion composition of the present disclosure can include a substantially anhydrous, oil-based carrier, a structurant, and a surfactant. The carrier may be an emollient, such as capric/caprylic triglyceride. The surfactant may be polypropylene glycol (such as PPG-15 stearyl ether) or PEO-4 polyethylene oxide. The structurant may be a fatty alcohol or fatty ester, such as behenyl alcohol.

The topsheet lotion composition may include a polypropylene glycol (“PPG”) material. In some examples, the lotion composition may consist essentially of a polypropylene glycol material, preferably a polypropylene glycol homopolymer such as polypropylene glycol, and optionally, a carrier. In other examples, the topsheet lotion may include a polypropylene glycol material selected from the group consisting of polypropylene glycol copolymer, polypropylene glycol surfactant, and mixtures thereof. The topsheet lotion including the polypropylene glycol material may serve to help reduce the adherence of menstrual fluid to the topsheet, and upon contact transfer of the topsheet lotion to the user/wearer, reduce the adherence of fluid to her skin, thereby reducing staining on the topsheet and reducing soiling of the skin. The topsheet lotion may also help to improve continuous fluid acquisition of the absorbent article.

The topsheet lotion may be applied in any known manner, in any known pattern, and to the wearer-facing surface of the topsheet 20. For example, the topsheet lotion may be applied in a pattern of generally parallel, longitudinally- or laterally-oriented stripes or bands. To avoid compressing or displacing any portion of the topsheet nonwoven or any three-dimensional features thereof, it may be desired that the topsheet lotion be applied via spraying (application of small droplets) discrete dots (obtained via, e.g., gravure printing), stripes that run in the longitudinal or lateral direction of the article, etc. In addition, or alternatively, the lotion composition can be disposed near channels or embossed areas when present in the absorbent article. With respect to applying the lotion composition to the outer surface (i.e., body facing surface) of the topsheet, it is believed that the lotion composition helps reduce the adherence of solid components of body fluids, such as menses, to the outer surface of the topsheet, thereby resulting in less staining on the topsheet of the absorbent article. A substantially uniformly sprayed application may be preferred. The atomized spraying may be done via any conventional means known in the art.

In some forms, the lotion composition may be provided in a target zone of an absorbent article. As shown in FIG. 1A, the target zone 25 of an absorbent article 10 represents the area of the absorbent article of expected body fluids insult. The absorbent article 10 is shown having an overall longitudinal length generally parallel to a Y-axis and an overall lateral width generally parallel to an X-axis. The absorbent article 10 further comprises a thickness in a Z-direction (not shown) which is perpendicular to an X-Y plane created by the X and Y axes.

In some forms, the target zone 25 may comprise about 50 percent of the overall longitudinal length (along a Y-axis) of the absorbent article 10 where each of the outer zones comprise about 25 percent or less of the overall length or less of the absorbent article 10. In some forms, the target zone 25 may comprise a surface area and the lotion composition may be on from about 30% to about 90% of the topsheet surface area, in some embodiments from about 50% to about 80% of the topsheet surface area. In some embodiments, from about 10% to about 40% of the lotion drops may be embedded in the topsheet, such as being completely surrounded by the topsheet fibers, that is, not resting on the surface of the topsheet. The lotion drops, or a majority of the lotion drops, may be on individual fibers of the topsheet. The lotion drops may be on both the outer surface (wearer-facing surface) and between the outer surface and inner surface of the topsheet.

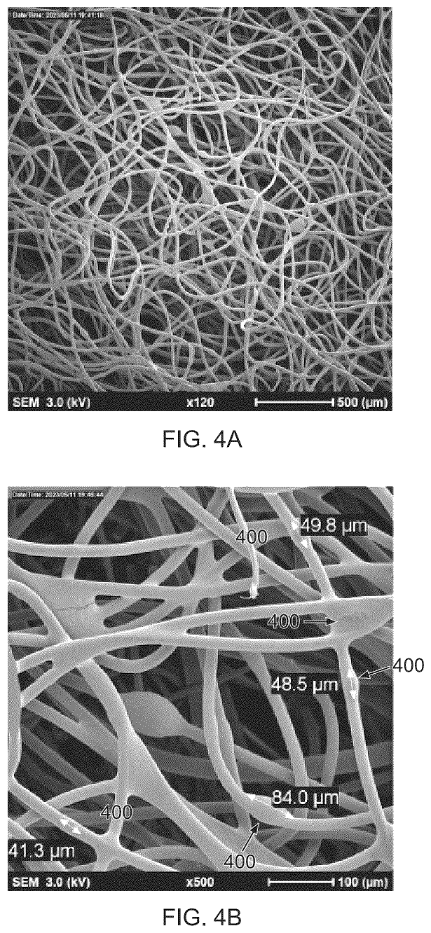

In some embodiments, the lotion composition is applied to the topsheet in discrete drops. Such drops may limit the material bridge between the fibers. In some embodiments, the width of each drop may be at most about 100 microns, in some embodiments, at most about 90 microns, in some embodiments at most about 80 microns. FIG. 4A and FIG. 4B are scanning electron micrograph (“SEM”) photos showing lotion composition example 1 (Table 1) and position profile in both cases include droplets (400 arrow), bridging between fibers (401 arrow) and extended droplets on single fiber. While not being bound by theory, it is believed that the lotion composition being applied in drops minimizes the amount of the lotion that is deposited as bridges between the fibers. When the lotion bridges between the fibers, it is less likely to be able to move the menses towards the absorbent core. But, as shown by the data herein, when the lotion composition is discrete drops on individual fibers, it is more effective at moving the fluid in the z-direction to reach the absorbent core, and thus minimizes the fluid left in the topsheet.

The lotion composition may be semi-solid or solid at room temperature, and liquid or semi-liquid at body temperature. It may be liquid with processible viscosity at temperature of 65-90° C.

The quantity of lotion composition applied may vary and can be adjusted for specific needs. For example, while not being bound by theory, it is believed that lotion composition may be applied at levels that are effective, of at least about 0.1 gsm, 0.5 gsm, 1 gsm, 2 gsm, 3 gsm, 4 gsm, 5 gsm, 10 gsm, up to about 15 gsm. It is believed that efficacy is not further enhanced above these upper limits, and so applications at basis weights exceeding these upper limits may be needless usage (waste) of lotion composition. The lotion composition can be applied within any subrange defined by any of the levels recited above (e.g., from about 0.1 gsm to about 15 gsm). These levels refer to the area of the topsheet surface to which the lotion composition is actually applied. It may be preferred that a majority, substantially all, or all of the surface area of the topsheet overlying the fluid management layer and/or absorbent core have the lotion composition applied. In certain embodiments, the minimum level of the lotion composition to be applied to a component of the absorbent article 10 can be an amount effective for reducing the adherence of menses to that component. This is because, as is believed, the lotion composition may enhance the ability of the topsheet to resist rewetting.

The lotion composition contemplated herein offers significant advantages over other lotion compositions, including non-PPG derived surfactants and other surface modifying agents. The advantages may be deemed particularly useful for feminine hygiene pads. Without intending to be bound by theory, it is believed that the superior fluid handling properties of the PPG materials identified herein is a result of the way in which the PPG materials act on the solid components of menstrual fluid, as opposed to surface energy treatments which act on the water component of menses. Surface energy treatments may be less effective due to the presence of polar and dispersive components in menstrual fluid, which may inhibit the effectiveness of surface energy treatments. Because the PPG materials identified herein are typically not readily soluble in menstrual fluid, they can effectively coat surfaces without dissolving in the fluid, which provides a hydrated barrier whose electron donating dipoles repel negatively dipoled proteins, thereby rendering the menstrual fluid less apt to adhere to surfaces of the article or the wearer's skin. Less adherence of menstrual fluid to the wearer's skin and/or to the topsheet promotes better and faster fluid movement through the topsheet, and fewer, smaller and/or less visible stain patterns on used products.

In some embodiments, it may be desired to include graphics provided by printing inks on the substrate materials by various printing methods, such as flexographic printing, rotogravure printing, screen-printing, inkjet printing, and the like. In some configurations, printing operations are performed separately to the assembly process, such as for example, printing the substrates offline, wherein the printed substrates may be stored until needed for production. For example, printing operations may be accomplished on discrete printing lines, separately from converting lines that are dedicated to manufacturing disposable absorbent articles.

However, inkjet printing operations utilized to print substrates, when incorporated into converting operations, is not without challenges in performing such printing processes and attempting to maintain aesthetically pleasing final assemblies. For example, multiple printheads may be needed to create graphics with multiple colors, relatively intricate designs, and/or relatively large sizes. In turn, it may be necessary to arrange multiple printheads along the machine direction and/or cross direction to enable printing at desired widths and/or with desired colors. In some configurations, when a design to be printed on a substrate is wider than the maximum print width of a single printhead, multiple printheads may be “stitched” or arranged together along the width of the substrate. As such, printheads may be arranged to print zones that are coterminous to one another along the cross direction to create a contiguous design. In some scenarios, the alignment between printed zones from different printheads may become undesirably offset in the machine direction and/or the cross direction. As such, the offset may result in a visibly noticeable stitch line in the printed region wherein the printed design appears disjointed, and in turn, may detract from aesthetically pleasing aspects of the printed regions. In addition, the aforementioned challenges may be exacerbated in absorbent article assembly processes operating at relatively high speed production rates and/or when printing on substrates that are extensible, such as nonwovens.

For lotion application in feminine hygiene pads that will have ink printed on substrates, the present inventors are aware that the printing and positioning of such ink graphics on substrates and/or components used in absorbent article assembly processes may be done in such a manner so as to functionally reduce noticeably visible results of imprecise and/or inconsistent printing operations. For example, while not being bound by theory, it is believed that the lotion composition may affect the adhesion of the inks to the surface of the fibers by affecting the wetting of the inks and or depositing over the lotion drops and not the fiber surface. One solution may be to do any ink printing step before a step applying the topsheet lotion. This can assure that the ink's adherence to the topsheet is not compromised or reduced by the lotion.

### Surfactant

The lotion composition contemplated herein may include a PPG material. PPG materials suitable for purposes contemplated herein include PPG homopolymer materials, PPG copolymer materials, and PPG surfactant materials, as well as mixtures thereof. The lotion composition may further comprise other optional ingredients. In one embodiment, the lotion composition consists essentially of, or consists of, a PPG material, preferably polypropylene glycol. In another embodiment, the lotion composition comprises a PPG material selected from the group consisting of polypropylene glycol copolymer, polypropylene glycol surfactant, and mixtures thereof.

The lotion composition contemplated herein may include a PPG material at a level of about 0.1% to 5%, by weight of the lotion composition. In some examples, the lotion composition may include less than about 5%, preferably from about 0.5% to 3%, and more preferably from about 1% to 2%, of a PPG material, by weight of the lotion composition.

Suitable PPG homopolymer materials may include those corresponding to the following formula:

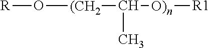

wherein R is hydrogen, methyl, ethyl, propyl, isopropyl, butyl, isobutyl, benzyl, aceto carbonyl, propio carbonyl, butyro carbonyl, isobutyro carbonyl, benzo carbonyl, fumaro carbonyl, aminobenzo carbonyl, carboxymethylene, aminopropylene, alkylated glucose, alkylated sucrose, alkylated cellulose, alkylated starch or phosphate; and wherein R is preferably hydrogen or methyl;
wherein R1 is hydrogen, methyl, ethyl, propyl, isopropyl, butyl, isobutyl, benzyl, aceto carbonyl, propio carbonyl, butyro carbonyl, isobutyro carbonyl, benzo carbonyl, fumaro carbonyl, aminobenzo carbonyl, carboxymethylene, aminopropylene, alkylated glucose, alkylated sucrose, alkylated cellulose, alkylated starch or phosphate; and wherein R1 is preferably hydrogen or methyl; and
wherein n is from 3 to 160, preferably from 5 to 120, more preferably from 10 to 100, and more preferably from 20 to 80.

Optionally, the PPG homopolymer may include low level of glycerol or butanediol as part of its monomer raw material. If included, the preferred ratio of glycerol or butanediol to propylene glycol may be 1:1000 to 1:2, most preferably 1:100 to 1:5. The PPG homopolymer may have, but is not necessarily limited to, CAS Numbers 25322-69-4, 25791-96-2 and 25231-21-4, wherein the latter is most preferred.

Non-limiting examples of suitable PPG homopolymer materials include polypropylene glycol 4000 such as Pluriol P-4000 (BASF), Alkapol PPG-4000 (Alkaril Chemical) and Niax Polyol PPG 4025 (Union Carbide); polypropylene glycol 3500; polypropylene glycol 3000 such as Niax PPG 3025 (Union Carbide); polypropylene glycol 2000 such as Alkanol PPG-2000 (Alkaril Chemical) and Pluriol P-2000 (BASF), polypropylene glycol 1200 such as Alkapol PPG-1200 (Alkaril Chemical) and Glucam P-20 Humectant (Noveon); polypropylene glycol 1000 such as Niax PPG 1025 (Union Carbide); polypropylene glycol 600 such as Alkanol PPG-600 (Alkaril Chemical) and Glucam P-10 Humectant (Noveon); polypropylene glycol 400 such as Alkanol PPG-425 (Alkaril Chemical). polypropylene glycol 4000 glycerol ether such as Pluriol T-4000 (BASF); polypropylene glycol 2000 glycerol ether, polypropylene glycol 2000 butanediol ether, polypropylene glycol 1500 glycerol ether such as Pluriol T-1500 (BASF), polypolypropylene glycol 4000 with monomethyl ether, polypropylene glycol 2000 with dimethyl ether, polypropylene glycol 4000 with monobutyl ether, polypropylene glycol 2000 with monobuytyl ether, polypropylene glycol 1200 with dibutyl ether, polypropylene glycol 4000 with bis(2-aminopropyl ether), PPG-10 methyl glucose ether and PPG-20 methyl glucose either.

Suitable PPG homopolymer materials will typically have a number average molecular weight of about 400 to 10,000, preferably about 600 to 6,000, and more preferably about 1,200 to 4,800.

Suitable PPG copolymer materials include those in which the polyprolyene glycol segments are present as an internal block component and/or as a terminal component, of the copolymer structure. The following formulae illustrate the internal block components and terminal block components:

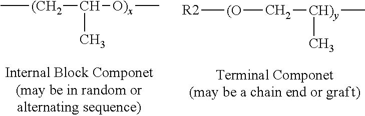

wherein x is 2 to 120, preferably 2 to 80, and more preferably 3 to 60; y is 2 to 100, preferably 2 to 50, and more preferably 3 to 30; R2 is hydrogen, methyl, ethyl, propyl, isopropyl, butyl, isobutyl, benzyl, aceto carbonyl, propio carbonyl, butyro carbonyl, isobutyro carbonyl, benzo carbonyl, fumaro carbonyl, aminobenzo carbonyl, carboxymethylene, aminopropylene, alkylated glucose, alkylated sucrose, alkylated cellulose, alkylated starch or phosphate, and wherein R2 is preferably hydrogen, methyl, ethyl, isopropyl or isobutyl.

Polymers suitable to form propoxylated copolymers with PPG for the present lotion composition s include homopolymers of alkyl methicone, phenyl methicone, dimethicone, alkyl trimethicone, phenyl trimethicone, polyol, polyether (e.g., polyoxymethylene, polyoxyethylene and polyoxypropylene), polyimine, polyamide, polyacrylate, polyester, and copolymers containing one or multiple of these polymeric units. Non-limiting examples of suitable polymers include polydimethyl siloxane, polyethylimine, polyacrylic acid, poly(ethylene-co-acrylic acid), polymethacrylic acid, poly(ethylene-co-methacrylic acid), poly(vinyl acetate), polyvinylpyrrolidone, poly(ethylene-co-vinyl acetate), poly(butanediol), poly(neopentyl glycol), poly(ethylene adipate), poly(butylene adipate), poly(ethylene glutamate), poly(butylene glutamate), poly(ethylene sebacate), poly(butylene sebacate), poly(ethylene succinate), poly(butylene succinate), poly(ethylene terephthalate), poly(butylene terephthalate), polycaprolactone, and polyglycerol.

Non-limiting examples of suitable PPG copolymer materials include PPG-12 dimethicone such as Sisoft 910 (Momentive); bis-PPG-15 dimethicon/IPDI copolymer such as Polyderm-PPI-SI-WI (Alzo); PPG/polycaprolactone block copolymer; PPG/polybutanediol/PEG triblock copolymer; polyethylimine/PPG copolymer and polyacrylic acid-g-PPG graft copolymer.

Another suitable PPG material includes PPG surfactant materials. The following formula represents suitable PPG surfactant materials wherein the PPG segments constitute a part of the head functional group:

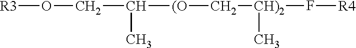

wherein R3 is hydrogen, alkyl, alkyl carbonyl, alkylenelamine, alkylenelamide, alkylene phosphate, alkylene carboxylic acid, alkylene sulfonate salt and alkylene quat with the maximum number of carbon element less than or equal to 6; R4 is octyl, nonyl, decyl, iosdecyl, lauryl, myristyl, cetyl, isohexadecyl, oleyl, stearyl, isostearyl, tallowoyl, linoleyl, jojoba, lanolin, behenyl, C24-C28 alkyl, C30-C45 alkyl, dinonylphenyl, dodecyl phenyl, or soya; z is from 1 to 100, preferably from 2 to 30, and more preferably from 3 to 25; and F is a functional group selected from the group consisting of ether groups (including oxy, glyceryl, glucose, sorbitan, sucrose, monoethanolamine or diethanolamine), ester groups (including ester, glyceryl ester, glucose ester, sorbitan ester or sucrose ester), amine groups, amide groups, and phosphate ester groups.

The following formula represents suitable PPG surfactant materials wherein the PPG segments constitute an internal block group:

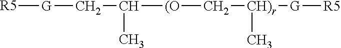

wherein R5 is hexyl, 2-ethylhexyl, octyl, nonyl, decyl, isodecyl, lauryl, cocoyl, myristyl, cetyl, isohexadecyl, oleyl, stearyl, isostearyl, tallow, linoleyl, octyl phenyl, or nonyl phenyl; r is from 1 to 120, preferably from 4 to 50, and more preferably from 6 to 30; and G is ether, ester, amine, or amide linkage.

Non-limiting examples of suitable PPG surfactant materials include PPG-30 cetyl ether such as Hetoxol C30P (Global Seven); PPG-20 methyl glucose ether distearate such as Glucam P-20 Distearate Emollient (Noveon), PPG-20 methyl glucose ether acetate, PPG-20 sorbitan tristearate, PPG-20 methyl glucose ether distearate, PPG-20 distearate, PPG-15 stearyl ether such as Alamol-E (Croda-Uniqema) and Procetyl 15 (Croda), PPG-15 stearyl ether benzoate, PPG-15 isohexadecyl ether, PPG-15 stearate, PPG-15 dicocoate, PPG-12 dilaurate, PPG-11 stearyl ether such as Varonic APS (Evonik); PPG-10 cetyl ether such as Procetyl 10 (Croda); PPG-10 glyceryl stearate, PPG-10 sorbitan monosterate, PPG-10 hydrogenated castor oil, PPG-10 cetyl phosphate, PPG-10 tallow amine, PPG-10 oleamide, PPG-10 cetyl ether phosphate, PPG-10 dinonylphenolate, PPG-9 laurate, PPG-8 dioctate, PPG-8 diethylhexylate, PPG-7 lauryl ether, PPG-5 lanolin wax ether, PPG-5 sucrose cocoate, PPG-5 lanolin wax, PPG-4 jojoba alcohol ether, PPG-4 lauryl ether, PPG-3 myristyl ether such as Promyristyl PM-3 (Croda), PPG-3 myristyl ether propionate such as Crodamol PMP (Croda), PPG-3 benzyl ether myristate such as Crodamol STS (Croda), PPG-3 hydrogenated castor oil such as Hetester HCP (Alzo), PPG-3-hydroxyethyl soyamide, PPG-2 Cocamide, PPG-2 lanolin alcohol ether and PPG-1 coconut fatty acid isopropanolamide such as Amizett IPC (Kawaken Fine Chemicals). A particularly preferred example of a suitable PPG material is PPG-15 stearyl ether, such as the product sold as CETIOL E, by BASF Corporation (Florham Park, New Jersey, USA) and/or BASF SE (Ludwigshafen, Germany).

### Carrier

Carriers suitable herein may include an emollient. In certain embodiments, these emollients will have either a semi-solid or liquid consistency at room temperatures, i.e., 20° C.

Suitable emollients can be derived from a renewable resource. For example, suitable emollients may be plant-derived emollients, derived from cultivated or uncultivated plants. Preferably, these plants are harvested and managed in a sustainable manner.

Other suitable emollients can include fatty acid ester type emollients, alkyl ethoxylate type emollients, fatty alcohol type emollients, and combinations thereof. Examples of each of these types of emollients (as well as others) are described in U.S. Pat. No. 6,570,054.

Other suitable carrier can include natural oils or fats, or natural oil or fat derivatives, in particular of plant origin. Non-limiting examples include shea butter, argan oil, oleic canola Oil (Brassica campestris, B. napus, B. rapa; characterized by having an oleic content greater than 70%, e.g., hi olcic canola oil, very high oleic canola oil, or partially hydrogenated canola oil), marula kernel oil (Sclerocarya birrea), palm oil (Elacis Guineensis Oil), palm olein, palm stearin, palm superolein, pecan oil, pumpkin seed oil, oleic safflower oil (Carthamus Tinctorius; characterized by having an oleic content of greater than about 30% and omega-6 fatty acid content of less than about 50%, e.g., hi oleic safflower oil), sesame oil (Sesamum indicum, S. orcintale), soybean oil (Glycine max, e.g., hi oleic soybean, low linolenic soybean oil, partially hydrogenated), olcic sunflower oil (Helianthus annus; characterized by having an oleic content of greater than about 40%, e.g., mid oleic sunflower or high oleic sunflower oil), apricot oil, babassu oil, castor oil, coconut oil, cod liver oil, hydrogenated corn oil, hydrogenated cottonseed oil, hazelnut oil, jojoba oil, macadamia oil, meadowfoam seed oil, mink oil, maringa oil, manila oil, mortierella oil, palm kernel oil, hydrogenated peanut oil, hydrogenated rapeseed oil, rose hip oil, hydrogenated safflower oil, hydrogenated soybean oil, hydrogenated sunflower oil, hydrogenated walnut oil, hydrogenated wheat germ oil, or the hardened derivatives thereof. Other non-limiting examples of fats and oils that are suitable carrier options herein include: butter, C12-C18 acid triglyceride, caprylic/capric/lauric triglyceride, caprylic/capric/linoleic triglyceride, caprylic/capric/stearic triglyceride, caprylic/capric triglyceride (mixture of C8/C10-between 70:30 to 50:50, including about 60:40), cocoa butter, C10-C18 triglycerides (carbon chain length from 10 to 18), egg oil, epoxidized soybean oil, glyceryl triacetyl hydroxystearate, glyceryl triacetyl ricinoleate, glycosphingolipids, hydrogenated castor oil, hydrogenated castor oil laurate, hydrogenated coconut oil, hydrogenated C12-C18 triglycerides, hydrogenated fish oil, hydrogenated lard, hydrogenated menhaden oil, hydrogenated mink oil, hydrogenated orange roughy oil, hydrogenated shark liver oil, hydrogenated tallow, hydrogenated vegetable oil, lanolin and lanolin derivatives, lanolin alcohol, lard, lauric/palmitic/oleic triglyceride, lesquerella oil, maleated soybean oil, neatsfoot oil, oleic/linoleic triglyceride, oleic/palmitic/lauric/myristic/linoleic triglyceride, oleostearine, olive husk oil, omental lipids, pengawar djambi oil, pentadesma butter, phospholipids, shea butter, tallow, tribchenin, tricaprin, tricaprylin, triheptanoin, trihydroxymethoxystearin, trihydroxystearin, triisononanoin, triisostearin, trilaurin, trilinolein, trilinolenin, trimyristin, trioctanoin, triolein, tripalmitin, trisebacin, tristearin, triundecanoin, and the like, as well as mixtures thereof. Oleic canola oil, palm oil, sesame oil, hi oleic safflower oil, hi oleic soybean oil, mid oleic sunflower oil, and high oleic sunflower oil are common plant-bred derived oils and may be also be derived from non-genetically modified organisms (non-GMO). Additional such emollients are also described in U.S. patent application Ser. No. 12/974,674.

In certain embodiments, the carrier can further comprise a blend of oils, including those described supra, as well as additional oil materials. Suitable additional emollients can include acai berry oil, almond oil, avocado oil, beech oil, brazil nut oil, camelina sativa oil (family Brassicaccac, e.g., Camelina Sativa, Gold of Pleasure, False Flax, etc.), camellia seed oil, canola oil, carrot seed oil, cashew nut oil, castor oil, cherry kernel oil, chia oil, corn oil, cottonseed oil, hydrogenated cottonseed oil, evening primrose oil, filbert (hazelnut) oil, grapeseed oil, hemp oil, hickory nut oil, jojoba oil, kukui oil, lanolin, olive oil (Olea europaca), macadamia oil, maringa oil, meadowfoam oil, neem oil, palm kernel oil, olive oil, passionflower oil (family Passiflora, Passiflora Incarnata), peanut oil, peach kernel oil, pistachio nut oil, rapeseed oil, rice bran oil, rose hip oil, safflower oil, sorghum oil, soybean oil, sunflower seed oil, tall oil, vegetable oil, vegetable squalene, walnut oil, wheat germ oil, and mixtures thereof. The oil material of the present invention can be selected from the group consisting of camelina sativa seed oil, oleic canola oil, evening primrose oil, manila kernel oil, palm oil, palm olein, palm stearin, palm superolein, Passiflora incarnata seed oil, pecan oil, pumpkin seed oil, oleic safflower oil, sesame oil, soybean oil, oleic sunflower oil, vegetable oil and mixtures thereof.

In certain embodiments, to further enhance the stability of the carrier, certain antioxidants can be added to certain emollients or to the lotion composition. In one embodiment, the carrier comprises from about 0.005% to about 1%, from about 0.01% to about 0.5%, or from about 0.02% to about 0.2%, by weight of the emollient, of an antioxidant. In one embodiment, the carrier composition comprises from about 0.0005% to about 1%, from about 0.001% to about 0.75%, or from about 0.002% to about 0.5%, by weight of the skin care composition, of an antioxidant. Non-limiting examples of suitable antioxidants include α-tocopherol, β-tocopherol, γ-tocopherol, δ-tocopherol, tocotrienol, rosemary, sesamol, sesamolin, sesamin, catechin, mixed tocopherols, citric acid, malic acid, grapeseed, green tea, mushroom extract, pine bark extract, licorice root, phytic acid, cranberry, pumpkin seed, wheat germ oil, and mixtures thereof.

Certain carriers may favor the transfer of the lotion composition from the absorbent article's wearer contacting surface to the skin. This may be the result of the size of the shape of the crystal structure that results from specific combinations of emollients and immobilization agents. Carrier and immobilizing agents that have similarities in their carbon chain structure may closely pack together into finer crystals, whereas emollients and immobilizing agents that have fewer similarities in their carbon chain structure, e.g., mixture of linear and branched carbon chains and/or mixture of linear and aromatic chains, may more loosely pack together into more amorphous crystals. Without being bound by theory, finer crystals with more edges or shape to the crystals may more readily transfer to the skin of the wearer through easier interaction with the skin through frictional forces relative to amorphous crystals.

The amount of the one or more emollient in the lotion composition may be from about 5% to about 99%, by weight of the lotion composition. In some embodiments, the lotion composition may comprise from about 40% to about 90%, from about 40% to about 80%, from about 50% to about 80%, from about 60% to about 80%, from about 60% to about 70%, or from about 70% to about 85%, by weight, of one or more emollient. The lotion compositions contemplated herein may include the carrier at a total carrier concentration ranging from about 60% to 99.9%, preferably about 70% to 99.5%, more preferably about 75% to 99% by weight of the lotion composition.

Other suitable carriers may include mono- or di-glycerides, such as those derived from saturated or unsaturated, linear or branch chained, substituted or unsubstituted fatty acids or fatty acid mixtures. Examples of mono- or diglycerides include mono- or di-C12-24fatty acid glycerides, specifically mono- or di-C16-20fatty acid glycerides, for example glyceryl monostearate, glyceryl distearate. Carriers can also include esters of linear C6-C22-fatty acids with branched alcohols.

Carriers contemplated herein may also include sterols, phytosterols, and sterol derivatives. Sterols and sterol derivatives that can be used in the lotion composition s of the invention include, but are not limited to: β-sterols having a tail on the 17 position and having no polar groups for example, cholesterol, sitosterol, stigmasterol, and ergosterol, as well as, C10-C30 cholesterol/lanosterol esters, cholecalciferol, cholesteryl hydroxystearate, cholesteryl isostearate, cholesteryl stearate, 7-dehydrocholesterol, dihydrocholesterol, dihydrocholesteryl octyldecanoate, dihydrolanosterol, dihydrolanosteryl octyldecanoate, ergocalciferol, tall oil sterol, soy sterol acetate, lanasterol, soy sterol, avocado sterols, “AVOCADIN” (trade name of Croda Ltd of Parsippany, N.J.), sterol esters and similar compounds, as well as mixtures thereof. A commercially available example of phytosterol is GENEROL 122 N PRL refined soy sterol from Cognis Corporation of Cincinnati, Ohio.

A particularly preferred example of a suitable carrier is caprylic/capric triglyceride. This material is currently available as, e.g., MYRITOL 318, a product of BASF Corporation (Florham Park, New Jersey, USA) and/or BASF SE (Ludwigshafen, Germany).

### Structurant/Rheological Agents

A lotion composition of the present invention comprises at least one rheology structurant, which typically is a solid. A “rheology structurant” is a material that helps immobilize lotion ingredients on the topsheet and impede migration through the topsheet into the absorbent core. If the lotion migrates to the absorbent core, the absorption properties of the core decrease, and more lotion must be applied to the topsheet in order to obtain a beneficial effect. The rheology structurant can also assist in transfer of a portion of the lotion to the skin and delivers target sensory at wearing. Typically, a rheology structurant rapidly solidifies on the top sheet after application of the lotion. The rheology structurant can be provided in an amount of about 0.1% to 95%, by weight of the lotion composition. In a preferred embodiment, the lotion compositions comprise about 1% to about 50% of a rheology structurant, by weight of the lotion composition. In a more preferred embodiment, lotion compositions comprise about 3% to about 30%, of a rheology structurant, by weight of the lotion composition. A preferred rheology structurant used in a present lotion composition comprises fatty alcohol having from about 12 to about 24 carbon atoms and/or a fatty monoester having from about 40 to about 44 carbon atoms.

Rheological agents can be added to the lotion composition. The carrier (e.g., the emollient) without the rheological agents exhibits typical Newtonian fluid characteristics. This drawback can lead to settling and bridging effects during processing and failure to apply the lotion composition to a substrate surface consistently beside the line hygiene problems. The rheology of the composition in its melt phase may be modified by an effective amount of the rheological agent(s) such that it behaves like a plastic or pseudoplastic fluid. The resultant lotion composition is a semi-solid/solid material. The stabilized composition is substantially free of agglomeration, stratification and/or settling; therefore, the melt composition can flow through processing equipment easily and be consistently applied to a substrate surface. It is found that both the clastic modulus and the apparent viscosity of the composition are factors affecting the processability of the lotion composition.

Specifically, the addition of a rheological agent can increase the elastic modulus of the lotion composition in certain embodiments to at least about 5 cP when measured at 80° C. under an oscillation frequency of 10 rad/sec and a shear strain of 0.2% (see test method disclosed herein). In other certain embodiments, the lotion composition can have an elastic modulus in the range from about 5 to about 25,000 dync/cm²; from about 10 to about 10,000 dyne/cm²; and from about 100 to about 5,000 dyne/cm². Examples of such rheological agents are described in U.S. Pat. No. 6,570,054.

The rheological properties (such as elastic modulus, viscosity) of the lotion composition in the melt form are measured using a viscometer (available from TA Instruments of New Castle, Del. as model number CSL 100) in an oscillation mode. The measurements are conducted using a conc-and-plate measuring system, having a diameter of 40 mm and a gap of 60 micron. The measurement commences after about 100 seconds waiting time. And the measurements are conducted at two temperatures: 80° C. and 40° C. The elastic modulus measured at 10 rad/sec frequency and 0.2% strain is used to characterize the compositions. That is, all the clastic moduli disclosed and/or claimed herein are measured at the operating conditions given above.

Certain embodiments of the lotion composition compositions can be solid, or more often semi-solid at room temperature, i.e., at 20° C. Being solid or semi-solid at room temperature, the lotion compositions do not have a tendency to flow and migrate to a significant degree to undesired locations of the article, and thus avoid significant interference with the absorbency of the article. This means less lotion composition is required for imparting desirable appearance, protective or conditioning benefits. In certain embodiments, lotion compositions of the present disclosure can have a zero shear viscosity at about 20° C. between about 1.0×10⁶ centipoise and about 1.0×10⁸ centipoise; in certain embodiments from between about 5.0×10⁶ centipoise and about 5.0×10⁷ centipoise; and in certain embodiments from between about 7.0×10⁶ centipoise and about 1.0×10⁷ centipoise. Generally, the value for “zero shear viscosity” can be obtained by extrapolating a viscosity versus shear rate plot to a shear rate of zero. However, for plastic or pseudoplastic fluids which exhibit a yield behavior at low shear rate, the extrapolation method often does not fully and accurately describe the material. Alternatively, the “zero shear viscosity” can be approximated by a viscosity measured at very low shear rates. As used herein, the term “zero shear viscosity” is the value measured by a cone and plate viscometer (available from TA Instruments of New Castle, Del. as model number CSL 100), at very low shear rates (e.g., 1.0 sec⁻¹ or lower) and at a temperature of about 20° C. A particularly preferred example of a suitable structurant is Behenyl Alcohol. This material is currently available as, e.g., LANETTE 22, a product of BASF Corporation (Florham Park, New Jersey, USA) and/or BASF SE (Ludwigshafen, Germany).

### Optional Skin Care Actives

In certain embodiments, the lotion composition may contain at least one skin care active. Such skin care actives may be insoluble or partially soluble solids in the substantially anhydrous, oil-based carrier. The skin care actives may be incorporated into the skin care composition, either directly or as a predispersion, with agitation.

Such skin care actives can include, but are not limited to, proton donating agents, protease and/or enzyme inhibitors, antimicrobials, humectants (glycerine, sorbitol), vitamins and derivatives thereof (e.g., Vitamins A, D, E and K), skin soothing and healing agents, such as aloe vera, or other ingredients from herbal, botanical or mineral sources, sunscreens, preservatives, anti-acne medicaments, antioxidants, chelators and sequestrants, essential oils, skin sensates, multi-functional agents, such as zinc oxide, and mixtures thereof. Examples of such skin care actives are described in U.S. Pat. No. 6,570,054 and U.S. patent application Ser. No. 12/974,674.

Lotions of the present invention facilitate fluid acquisition through the top sheet to the absorbent core of an absorbent article, and resist fouling of the topsheet and the skin of the wearer by the proteins, lipids, and carbohydrates present in menses. Non-limiting examples of topsheet lotion compositions are illustrated in the following table, Examples 1-5.

| Materials | | | Weight % | | | | |
|---|---|---|---|---|---|---|---|
| | Trade | | Example | Example | Example | Example | Example |
| Component | Name | Supplier | Lotion 1 | Lotion 2 | Lotion 3 | Lotion 4 | Lotion 5 |
| Caprylic/Capric | Myritol | BASF | 89 | 88 | 49 | 24 | 9 |
| Triglyceride | 318 | | | | | | |
| Behenyl Alcohol | Lanette 22 | BASF | 10 | 10 | 50 | 75 | 90 |
| PPG-15 stearyl | Cetiol E | BASF | 1 | 2 | 1 | 1 | 1 |
| ether | | | | | | | |
| Viscosity at 80° C. | | | 3.1 cP | 3.2 cP | 3.2 cP | 3.5 cP | 4.1cp |

### Absorbent Core

### Absorbent Structure

The absorbent structure 40 of the present disclosure may have any suitable shape including but not limited to oval, a stadium, rectangle, an asymmetric shape, peanut, trapezoid, rounded trapezoid, ovoid, and hourglass. In some examples, absorbent structure 40 may have a contoured shape, e.g., one that is narrower in the longitudinally intermediate region than in the end regions. In other examples, the absorbent structure may have a tapered shape that is a wider in one end region of the pad, and tapers to a narrower width in the other end region of the pad. The absorbent structure 40 may have varying stiffnesses in the MD and CD.

The configuration and construction of the absorbent structure 40 may vary (e.g., the absorbent structure 40 may have varying caliper zones, a hydrophilic gradient, a superabsorbent gradient, or lower average density and lower average basis weight acquisition zones). Further, the size and absorbent capacity of the absorbent structure 40 may also be varied to accommodate a variety of wearers. However, the total absorbent capacity of the absorbent structure 40 should be compatible with the design loading and the intended use of the disposable absorbent article or incontinence pad 10.

In some examples, the absorbent structure 40 may include a plurality of layers each having particular features and/or functions. In some examples, the absorbent structure 40 may include a wrap (not shown) included to envelope the absorbent constituents of the absorbent structure. The wrap may be formed by one or more nonwoven materials, tissues, films or other materials, or laminates thereof. In one form, the wrap may be formed of only a single material, substrate, laminate, or other material that is wrapped at least partially around itself.

The absorbent structure 40 may include one or more adhesives, for example, to help immobilize the SAP or other absorbent materials within the first and second laminates.

Suitable absorbent structures comprising relatively high amounts of superabsorbent polymer (“SAP”—also known as “absorbent gelling material,” or “AGM”) with various core designs are disclosed in U.S. Pat. No. 5,599,335; EP 1 447 066; WO 95/11652; US 2008/0312622A1; and WO 2012/052172

Additions to the absorbent structure are contemplated. Potential additions to the absorbent structure are described in U.S. Pat. Nos. 4,610,678; 4,673,402; 4,888,231; and 4,834,735. The absorbent structure may further include layers that mimic the dual core system containing an acquisition/distribution core of chemically stiffened fibers positioned over an absorbent storage core as described in U.S. Pat. No. 5,234,423; and in U.S. Pat. No. 5,147,345. These may be deemed useful to the extent they do not negate or conflict with the effects of the below described laminates of the absorbent structure of the present invention.

Some further examples of a suitable absorbent structures 40 that can be used in the absorbent article of the present disclosure are described in US 2018/0098893 and US 2018/0098891.

As noted above, absorbent articles including a fluid management layer contemplated herein may include a storage layer. Referring back to FIGS. 1A and 1B, a storage layer would generally be positioned at a location corresponding to that in which the absorbent structure 40 is depicted. The storage layer may be constructed as described regarding absorbent structures. The storage layer may contain conventional absorbent materials. In addition to conventional absorbent materials such as creped cellulose wadding, fluffed cellulose fibers, rayon fibers and comminuted wood pulp fibers (also known as airfelt or fluff pulp), and textile fibers, the storage layer may also include particles or fibers of superabsorbent material that imbibes fluids and forms hydrogels. (Such materials are also known as absorbent gelling materials (AGM).) AGM is typically capable of absorbing a relatively large weight quantity of body fluid per dry weight AGM, retaining it under moderate pressure. Synthetic fibers spun from polymers such as cellulose acetate, polyvinyl fluoride, polyvinylidene chloride, acrylics (such as ORLON), polyvinyl acetate, non-soluble polyvinyl alcohol, polyethylene, polypropylene, polyamides (such as nylon), polyesters, bi-component fibers, tricomponent fibers, mixtures thereof and the like can also be included in the secondary storage layer. The storage layer may also include filler materials, such as PERLITE, diatomaceous earth, VERMICULITE, or other suitable materials, that can serve to reduce changes of rewetting.

Fluid management layers contemplated herein may be incorporated into a variety of absorbent articles. A non-limiting example of a schematic representation of an absorbent article in the form of a feminine hygiene pad as contemplated herein is shown in FIG. 1A. As reflected, the pad 10 as contemplated herein may include a topsheet 20, a backsheet 50, and an absorbent structure 40 disposed between the topsheet 20 and the backsheet 50. A fluid management layer 30 may be disposed between the topsheet 20 and the absorbent structure 40. The pad has a wearer-facing surface 60 and an opposing outward-facing surface 62. The wearer-facing surface 60 is formed primarily by the topsheet 20 while the outward-facing surface 62 is formed primarily by the backsheet 50. Additional components (not shown) may be included proximate the wearer-facing surface 60 and/or the outward-facing surface 62. For example, if the absorbent article is an incontinence pad, a pair of barrier cuffs which extend generally parallel to a longitudinal axis 100 of the pad 10 and may also form portions of the wearer-facing surface 60. Similarly, one or more deposits fastening adhesive (to be used by the user/wearer to affix the pad within her underwear, at an appropriate location, for use) may be present on the backsheet 50 and form a portion of the outward-facing surface 62 of the absorbent article.

A non-limiting example of a configuration for the fluid management layer 30 is schematically depicted in FIG. 1B. As reflected, the fluid management layer 30 may have opposing end edges 32A and 32B which may extend generally parallel to a lateral axis 200, and side edges 31A and 32B that may extend generally parallel to the longitudinal axis 100. Similarly, the absorbent structure 40 may have opposing end edges 42A and 42B which may extend generally parallel to the lateral axis 200, and side edges 41A and 41B that may extend generally parallel to the longitudinal axis 100.

As reflected in the figures, each of the end edges 32A and 32B of the fluid management layer 30 may be disposed longitudinally outboard of the absorbent structure 40. However, this is not necessarily required. For example, the end edges 32A and/or 32B may be coextensive with the absorbent structure 40 or the end edges 32A and/or 32B may be disposed longitudinally inboard of the end edges 42A and/or 42B of the absorbent structure 40.

Similarly, the side edges 31A and/or 31B may be disposed laterally outboard of the side edges 41A and/or 41B of the absorbent structure 40. Alternatively, the side edges 31A and/or 31B may be laterally coextensive with the side edges 41A and/or 41B of the absorbent structure 40.

The storage layer or fluid storage layer may include absorbent gelling material (AGM) in a uniform distribution throughout, or may include it in a non-uniform distribution. The AGM may be distributed and/or concentrated via deposit thereof into channels or pockets, or may be deposited in patterns including stripes, crisscross patterns, swirls, dots, or any other pattern, either two or three dimensional, that can be imagined. The AGM may be sandwiched between a pair of fibrous cover layers. AGM may be encapsulated, at least in part, by a single fibrous cover layer.

Portions of the storage layer may be formed substantially only of superabsorbent material/AGM, or may be formed of AGM distributed and dispersed in a suitable carrier structure such as a batt or accumulation of cellulose fibers in the form of fluff or stiffened fibers. One non-limiting example of a storage layer may include a first layer formed substantially only of AGM particles or fibers, that are placed or deposited onto a second layer that is formed of a distribution of AGM particles or fibers, within cellulose fibers.

Examples of absorbent structures formed of layers of superabsorbent material/AGM and/or layers of superabsorbent material/AGM dispersed within a batt or other accumulation of cellulose fibers, that may be utilized in the absorbent articles (e.g., sanitary napkins, incontinence products) contemplated herein are disclosed in US 2010/0228209A1. Absorbent structures comprising relatively high amounts of SAP/AGM with various core designs are disclosed in U.S. Pat. No. 5,599,335; EP 1 447 066; WO 95/11652; US. 2008/0312622A1; WO 2012/052172; U.S. Pat. No. 8,466,336; and 9,693,910 to Carlucci. These may be used to configure the absorbent structure or storage layer.

### Backsheet

The backsheet 50 may be disposed beneath the absorbent structure 40 and be the outwardmost layer of the article, forming the outward-facing surface of the article. The backsheet 50 may be joined to the absorbent structure 40 and/or to the topsheet (about the outer periphery) by any suitable attachment methods known in the art. For example, the backsheet 50 may be secured to the absorbent structure 40 by a uniform continuous layer of adhesive, a patterned layer of adhesive, or an array of separate lines, spirals, or spots of adhesive. Alternatively, the attachment methods may comprise using heat bonds, pressure bonds, ultrasonic bonds, dynamic mechanical bonds, or any other suitable attachment methods or combinations of these attachment methods as are known in the art.

The backsheet 50 may be impervious, or substantially impervious, to liquids (e.g., urine, menstrual fluid) under ordinary conditions of use, and may be manufactured from a thin plastic film, although other flexible liquid impervious materials may also be used. The backsheet 50 may prevent, or at least inhibit, exudates absorbed and contained in the absorbent structure 40 from wetting underwear, outer clothing, bedding, etc. which may come into contact with or proximity to the article 10. However, in some examples the backsheet 50 may be configured so as permit vapor to escape from the absorbent structure 40 (i.e., is “breathable”) while in examples the backsheet 50 may be configured so as to be vapor-impermeable (i.e., non-breathable). Backsheet 50 may include a polymeric film such as a film of polyethylene or polypropylene. A suitable material for the backsheet 50 is a thermoplastic film having a thickness of approximately 0.012 mm (0.5 mil) to 0.051 mm (2.0 mils), for example. Any suitable liquid impermeable backsheet known in the art may be utilized with the present invention.

The backsheet 50 serves as a barrier to prevent migration of fluids absorbed and retained in the absorbent structure 40, to the outward-facing surface of the pad. A preferred material is a soft, smooth, compliant, liquid and vapor pervious material that provides for softness and conformability for comfort, and is low noise producing so that movement does not cause unwanted sound.

Non-limiting examples of materials suitable for forming backsheets are described in U.S. Pat. Nos. 5,885,265; 6,462,251; 6,623,464; and 6,664,439. Examples of suitable dual- or multi-layer breathable backsheets include those described in U.S. Pat. Nos. 3,881,489; 4,341,216; 4,713,068; 4,818,600; EP 203 821; EP 710 471; EP 710 472; and EP 793 952. Additional examples of suitable single layer breathable backsheets for include those described in GB A 2184 389; GB A 2184 390; GB A 2184 391; U.S. Pat. Nos. 4,591,523; 3,989,867; 3,156,242; and WO 97/24097.

The backsheet may be a nonwoven web having a basis weight of about 20 gsm to 50 gsm. In one example, the backsheet may be a hydrophobic 23 gsm spunbond nonwoven web of 4 denier polypropylene fibers, available from Fiberweb Neuberger, under the trade designation F102301001. The backsheet may be coated with a non-soluble, liquid swellable material as described in U.S. Pat. No. 6,436,508.

The backsheet has an outward-facing side and an opposing wearer-facing side. The outward-facing side of the backsheet may include a non-adhesive area and an adhesive area. The adhesive area may be provided by any conventional means, for the purpose of enabling the user/wearer to affix the pad to the wearer-facing surface of her underwear at a location suitable for use. Pressure-sensitive adhesives have been found to work well for this purpose.

### Experimentation

For purposes of experimentation and discovery, the inventors made and tested various samples of absorbent articles in the form of feminine hygiene pads, including various examples of lotion compositions and topsheets as contemplated herein. The prototype samples 1-8 had the following features:

### Topsheets

Topsheets for all prototype samples had a basis weight of about 24 gsm.

Topsheets for all prototype samples were cut from a nonwoven web of carded staple length fibers having an average staple length of 38 mm and an average denier of 4. The fibers were bicomponent, with a concentric sheath-core configuration, in which the core component was PET and the sheath component was low density polyethylene (LDPE); the weight ratio of the components was approximately 1:1. The nonwoven web included a plurality of randomly-distributed fiber-to-fiber bonds that had been created via air-through heat bonding of the carded fibers.

The topsheets were affixed to the subjacently-disposed fluid management layers via application of a pressure sensitive adhesive applied in a discontinuous series of narrow spiral paths generally oriented in the longitudinal direction. The fluid management layers were affixed to the subjacently-disposed absorbent structures in a similar manner.

Samples 1 and 2 are control samples, not having the topsheet lotion composition in drops on the topsheet. Samples 3-8 all are inventive examples, as they have drops of Example Lotions on the topsheet. Further details varied among samples as specified below:

Sample #1: The topsheet nonwoven was formed of a blend of hydrophilic and hydrophobic fibers, in a ratio of 60:40, weight of hydrophilic fibers to weight of hydrophobic fibers. The topsheet nonwoven did not include apertures and did not have any topsheet lotion composition.

Sample #2: The topsheet nonwoven was formed of a blend of hydrophilic and hydrophobic fibers, in a ratio of 60:40, weight of hydrophilic fibers to weight of hydrophobic fibers. The topsheet nonwoven included a pattern of apertures having an average x-y dimension aperture area of 0.55 mm², and constituting an open area of approximately 6 percent. The topsheet nonwoven did not have any topsheet lotion composition.

Sample #3: The topsheet nonwoven was formed of a blend of hydrophilic and hydrophobic fibers, in a ratio of 60:40, weight of hydrophilic fibers to weight of hydrophobic fibers. The topsheet nonwoven did not include apertures. The pads comprise Example Lotion 1, having been sprayed on to the wearer-facing surface at an application level of about 2 gsm.

Sample #4: The topsheet nonwoven was formed of a blend of hydrophilic and hydrophobic fibers, in a ratio of 60:40, weight of hydrophilic fibers to weight of hydrophobic fibers. The topsheet nonwoven did not include apertures. The pads contain Example Lotion 2, having been sprayed on to the wearer-facing surface at an application level of about 2 gsm.

Sample #5: The topsheet nonwoven was formed of a blend of hydrophilic and hydrophobic fibers, in a ratio of 60:40, weight of hydrophilic fibers to weight of hydrophobic fibers. The topsheet nonwoven did not include apertures. The pads contain Example Lotion 3, having been sprayed on to the wearer-facing surface at an application level of about 2 gsm.

Sample #6: The topsheet nonwoven was formed of a blend of hydrophilic and hydrophobic fibers, in a ratio of 60:40, weight of hydrophilic fibers to weight of hydrophobic fibers. The topsheet nonwoven did not include apertures. The pads contain Example Lotion 4, having been sprayed on to the wearer-facing surface at an application level of about 2 gsm.

Sample #7: The topsheet nonwoven was formed of a blend of hydrophilic and hydrophobic fibers, in a ratio of 60:40, weight of hydrophilic fibers to weight of hydrophobic fibers. The topsheet nonwoven did not include apertures. The pads contain Example Lotion 5, having been sprayed on to the wearer-facing surface at an application level of about 2 gsm.

Sample #8: The topsheet nonwoven was formed of a blend of hydrophilic and hydrophobic fibers, in a ratio of 60:40, weight of hydrophilic fibers to weight of hydrophobic fibers. The topsheet nonwoven included a pattern of apertures having an average x-y dimension aperture area of 0.55 mm² and constituting an open area of approximately 6 percent. The pads contain Example Lotion 1, having been sprayed on to the wearer-facing surface at an application level of about 2 gsm.

### Fluid Management Layer

All prototype samples included a fluid management layer of a substantially common structure and composition, disposed subjacent the topsheet. The common fluid management layer had a basis weight of about 65 gsm. It was constituted of about 20 percent by weight, 1.3 dtex viscose fibers; about 30 percent by weight, 10 dtex hollow spiral polyethylene terephthalate fibers; and about 50 percent by weight, 2.2 dtex bi-component fibers having a concentric sheath-core configuration, wherein the core component was PET and the sheath component was PE, in a weight ratio of PET:PE of about 1:1. These bicomponent fibers had an average decitex of about 2.2. The fluid management layer had two strata each having the same homogeneous blend of fibers, was lightly hydroentangled, and was air-through heat bonded.

### Absorbent Structure

All prototype samples included an absorbent structure of a substantially common structure and composition, disposed subjacent the fluid management layer. The common absorbent structure was an airlaid blend of pulp fibers, absorbent gelling material, and bicomponent fibers, having a basis weight of 182 gsm, available pre-manufactured in festooned web form from Glatfelter Corporation (Charlotte, North Carolina, USA). It is not believed that the structure and composition of the absorbent structure had any significant role in the differences in performance measured among the various prototype samples.

### Backsheet

All prototype samples included a common backsheet disposed beneath the absorbent structure, formed from a sheet of extruded polyethylene film.

### Test Results and Data

Quantities of all 8 prototype samples were subjected to measurement and testing using the Acquisition Time and Rewet Measurement Method set forth below.

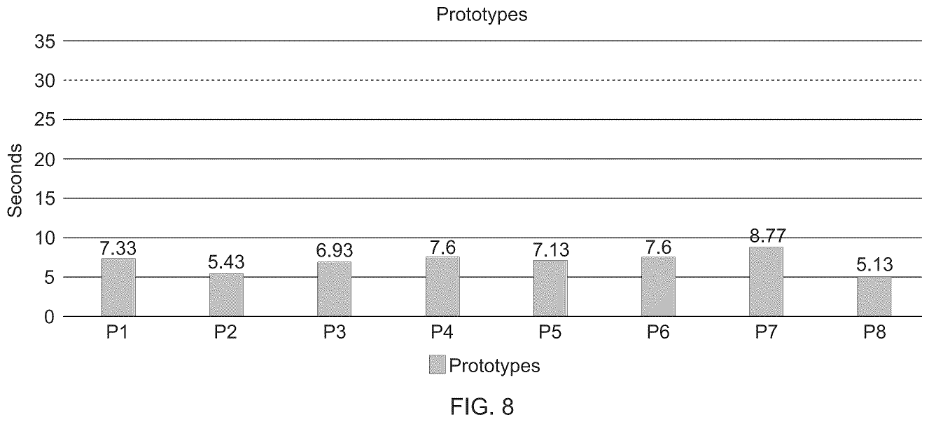

FIG. 8 reports the second acquisition time (ACQ-2) measured for each of the 8 samples. For purposes herein, it is believed that the second acquisition time (ACQ-2) is most relevant to the actual user experience because it reflects fluid acquisition for a feminine hygiene pad that has been in use/worn for some duration and has absorbed some fluid, but is then exposed to a relatively larger discharge of fluid as might occur with a sudden change of the user's/wearer's body position following a period of inactivity or relatively low activity. The heavy horizontal line drawn at the 30-second mark in the charts represents what the inventors believe to be the highest acquisition time that is acceptable to the relevant consumers/users.

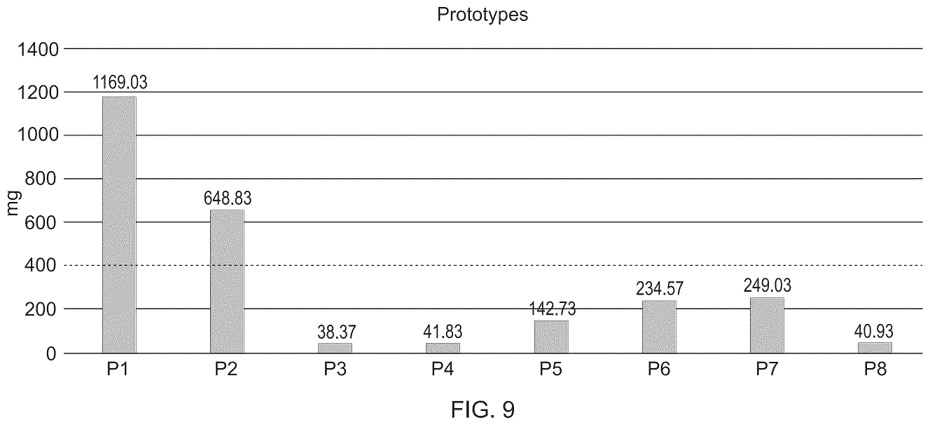

FIG. 9 reports the sum of surface free fluid (SFF) and rewet measured for each of the 8 samples. For purposes herein, it is believed that this sum is most relevant to the actual user experience because it reflects the extent to which a pad that has absorbed a substantial quantity of fluid will permit the fluid to return to the surface of the topsheet under moderate pressure, which is the cause of an unsatisfactory wet feeling for the user/wearer. The heavy horizontal line drawn at the 400-milligram mark in the charts represents what the inventors believe to be the highest SFF+Rewet that is acceptable to the relevant consumers/users.

From the chart in FIG. 8, it can be seen that all prototype samples exhibited second acquisition times that are deemed acceptable. Prototype samples 2, 3, and 5 exhibited good performances, with prototype sample 8 exhibiting the best performance of all.

From the chart in FIG. 9, it can be seen that all inventive examples (P3 to P8) exhibited SFF+Rewet values that are deemed acceptable, while the control samples (P1 and P2) did not exhibit acceptable values. Prototype sample 3 exhibited the best performance of all, followed by prototype samples 4, and 8, in that order.

From this data it might be concluded that prototype sample 3 was the most successful, as it appears among the top four performers in both categories. In varying circumstances, however, the product designer might choose to prioritize rapid acquisition (e.g., prototype sample 8 was the best performer among the test samples), or alternatively, low rewet value (e.g., prototype sample 3 was the best performer among the test samples).

In view of the description above, the following non-limiting embodiments are contemplated. Any of these embodiments as well as others may be claimed in one or more subsequent non-provisional patent applications based in whole or in part on the disclosure herein:
1. The feminine hygiene pad of any of the embodiments herein, wherein the topsheet bears a topical application of a lotion composition.
2. The feminine hygiene pad of embodiment 1 wherein the lotion composition comprises a polypropylene glycol material, a carrier, and structurant rheology modifier.
3. The feminine hygiene pad of embodiment 2 wherein the polypropylene glycol material is selected from the group consisting of polypropylene glycol copolymer, polypropylene glycol surfactant, and mixtures thereof.
4. The feminine hygiene pad of embodiment 3 wherein the polypropylene glycol material is polypropylene glycol copolymer, and wherein said polypropylene glycol copolymer comprises an internal block component and a terminal block component, wherein said internal block component has a formula:

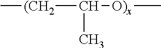

and said terminal block component has a formula:

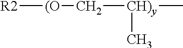

wherein x is from 2 to 120, y is from 2 to 100, and R2 is hydrogen, methyl, ethyl, propyl, isopropyl, butyl, isobutyl, benzyl, aceto carbonyl, propio carbonyl, butyro carbonyl, isobutyro carbonyl, benzo carbonyl, fumaro carbonyl, aminobenzo carbonyl, carboxymethylene, aminopropylene, alkylated glucose, alkylated sucrose, alkylated cellulose, alkylated starch or phosphate.
5. The feminine hygiene pad of any one of the preceding embodiments wherein the polypropylene glycol material is polypropylene glycol copolymer, and wherein said polypropylene glycol copolymer is selected from the group consisting of PPG-12 dimethicone, bis-PPG-15 dimethicone/IPDI copolymer, PPG/polycaprolactone block copolymer, PPG/polybutanediol/PEG triblock copolymer, polyethylimine/PPG copolymer; polyacrylic acid-g-PPG graft copolymer, and mixtures thereof.
6. The feminine hygiene pad of any one of the preceding embodiments wherein the polypropylene glycol material is polypropylene glycol surfactant, and wherein said polypropylene glycol surfactant has a formula:

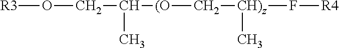

wherein R3 is hydrogen, alkyl, alkyl carbonyl, alkylenelamine, alkylenelamide, alkylene phosphate, alkylene carboxylic acid, alkylene sulfonate salt or alkylene quat with a maximum number of carbon elements of less than or equal to 6; R4 is octyl, nonyl, decyl, iosdecyl, lauryl, myristyl, cetyl, isohexadecyl, oleyl, stearyl, isostearyl, tallowoyl, linoleyl, jojoba, lanolin, behenyl, C24-C28 alkyl, C30-C45 alkyl, dinonylphenyl, dodecyl phenyl, or soya; z is from 1 to 100; and F is a functional group selected from the group consisting of an ether group, an ester group, an amine group, an amide group, and a phosphate ester group.
7. The feminine hygiene pad of any one of the preceding embodiments, wherein the polypropylene glycol material is polypropylene glycol surfactant, and wherein said polypropylene glycol surfactant has a formula:

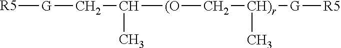

wherein R5 is hexyl, 2-ethylhexyl, octyl, nonyl, decyl, isodecyl, lauryl, cocoyl, myristyl, cetyl, isohexadecyl, oleyl, stearyl, isostearyl, tallow, linoleyl, octyl phenyl, or nonyl phenyl; r is from 1 to 120; and G is an ether, an ester, an amine, or an amide linkage.
8. The feminine hygiene pad of any one of the preceding embodiments wherein the polypropylene glycol material is polypropylene glycol surfactant, and wherein said polypropylene glycol surfactant is selected from the group consisting of PPG-30 cetyl ether, PPG-20 methyl glucose ether distearate, PPG-20 methyl glucose ether acetate, PPG-20 sorbitan tristearate, PPG-20 methyl glucose ether distearate, PPG-20 distearate, PPG-15 stearyl ether, PPG-15 stearyl ether benzoate, PPG-15 isohexadecyl ether, PPG-15 stearate, PPG-15 dicocoate, PPG-12 dilaurate, PPG-11 stearyl ether, PPG-10 cetyl ether, PPG-10 glyceryl stearate, PPG-10 sorbitan monosterate, PPG-10 hydrogenated castor oil, PPG-10 cetyl phosphate, PPG-10 tallow amine, PPG-10 oleamide, PPG-10 cetyl ether phosphate, PPG-10 dinonylphenolate, PPG-9 laurate, PPG-8 dioctate, PPG-8 diethylhexylate, PPG-7 lauryl ether, PPG-5 lanolin wax ether, PPG-5 sucrose cocoate, PPG-5 lanolin wax, PPG-4 jojoba alcohol ether, PPG-4 lauryl ether, PPG-3 myristyl ether, PPG-3 myristyl ether propionate, PPG-3 benzyl ether myristate, PPG-3 hydrogenated castor oil, PPG-3-hydroxyethyl soyamide, PPG-2 cocamide, PPG-2 lanolin alcohol ether, PPG-1 coconut fatty acid isopropanolamide, and mixtures thereof.
9. The feminine hygiene pad of any one of the preceding embodiments or of examples 3-8 wherein the polypropylene glycol surfactant is PPG-15 stearyl ether.
10. The feminine hygiene pad of any one of the preceding embodiments or of examples 3-8 wherein the polypropylene glycol material is polypropylene glycol homopolymer, and wherein said polypropylene glycol homopolymer has a formula:

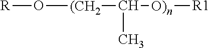

wherein R is hydrogen, methyl, ethyl, propyl, isopropyl, butyl, isobutyl, benzyl, aceto carbonyl, propio carbonyl, butyro carbonyl, isobutyro carbonyl, benzo carbonyl, fumaro carbonyl, aminobenzo carbonyl, carboxymethylene, aminopropylene, alkylated glucose, alkylated sucrose, alkylated cellulose, alkylated starch or phosphate; R1 is hydrogen, methyl, ethyl, propyl, isopropyl, butyl, isobutyl, benzyl, aceto carbonyl, propio carbonyl, butyro carbonyl, isobutyro carbonyl, benzo carbonyl, fumaro carbonyl, aminobenzo carbonyl, carboxymethylene, aminopropylene, alkylated glucose, alkylated sucrose, alkylated cellulose, alkylated starch or phosphate; and n is from 3 to 160.
11. The feminine hygiene pad of any one of the preceding embodiments or of examples 3-8 wherein the polypropylene glycol material is polypropylene glycol.
12. The feminine hygiene pad of any one of the preceding embodiments or of example 3-8 wherein the polypropylene glycol has a number average molecular weight of from about 400 to about 10,000.
13. The feminine hygiene pad of any one of the preceding embodiments or of any of examples 3-8 wherein the carrier comprises caprylic/capric triglyceride.

### Test and Measurement Methods

### Caliper

The caliper, or thickness, of a test specimen is measured as the distance between a reference platform on which the specimen rests and a pressure foot that exerts a specified amount of pressure onto the specimen over a specified amount of time. All measurements are performed in a laboratory maintained at 23° C.±2° C. and 50%±2% relative humidity and test specimens are conditioned in this environment for at least 2 hours prior to testing.

Caliper is measured with a manually-operated micrometer equipped with a pressure foot capable of exerting a steady pressure of 0.50 kPa±0.01 kPa onto the test specimen. The manually-operated micrometer is a dead-weight type instrument with readings accurate to 0.01 mm. A suitable instrument is Mitutoyo Series 543 ID-C Digimatic, available from VWR International, or equivalent. The pressure foot is a flat ground circular movable face with a diameter that is smaller than the test specimen and capable of exerting the required pressure. A suitable pressure foot has a diameter of 25.4 mm, however a smaller or larger foot can be used depending on the size of the specimen being measured. The test specimen is supported by a horizontal flat reference platform that is larger than and parallel to the surface of the pressure foot. The system is calibrated and operated per the manufacturer's instructions.

Obtain a test specimen by removing it from an absorbent article, if necessary. When excising the test specimen from an absorbent article, use care to not impart any contamination or distortion to the test specimen layer during the process. The test specimen is obtained from an area free of folds or wrinkles, and it must be larger than the pressure foot.

To measure caliper, first zero the micrometer against the horizontal flat reference platform. Place the test specimen on the platform with the test location centered below the pressure foot. Gently lower the pressure foot with a descent rate of 3.0 mm±1.0 mm per second until the full pressure is exerted onto the test specimen. Wait 5 seconds and then record the caliper of the test specimen to the nearest 0.001 mm. In like fashion, repeat for a total of ten replicate test specimens. Calculate the arithmetic mean for all caliper measurements and report as Caliper to the nearest 0.001 mm.

### Caliper Factor

The caliper factor, as mentioned previously is the caliper per 10 gsm of basis weight of the sample. So, the equation is caliper/(basis weight/10).

### Basis Weight

The basis weight of a sample of sheet or web material is the mass (in grams) per unit area (in square meters) of a single layer of the material. If it is not otherwise known or available, basis weight may be measured using EDANA compendial method NWSP 130.1. The mass of the test sample is cut to a known area, and the mass of the sample is determined using an analytical balance accurate to 0.0001 grams. All measurements are performed in a laboratory maintained at 23° C.±2° C. and 50%±2% relative humidity and test samples are conditioned in this environment for at least 2 hours prior to testing.

Measurements are made on test samples taken from rolls or sheets of the raw material, or test samples obtained from a material layer removed from an absorbent article. When excising the material layer from an absorbent article, use care to not impart any contamination or distortion to the layer during the process. The excised layer should be free from residual adhesive. To ensure that all adhesive is removed, soak the layer in a suitable solvent that will dissolve the adhesive without adversely affecting the material itself. One such solvent is THF (tetrahydrofuran, CAS 109-99-9, for general use, available from any convenient source). After the solvent soak, the material layer is allowed to thoroughly air dry in such a way that prevents undue stretching or other deformation of the material. After the material has dried, a test specimen is obtained. The test specimen must be as large as possible so that any inherent material variability is accounted for.

Measure the dimensions of the single layer test specimen using a calibrated steel metal ruler traceable to NIST, or equivalent. Calculate the Area of the test specimen and record to the nearest 0.0001 square meter. Use an analytical balance to obtain the Mass of the test specimen and record to the nearest 0.0001 gram. Calculate Basis Weight by dividing Mass (in grams) by Area (in square meters) and record to the nearest 0.01 grams per square meter (gsm). In like fashion, repeat for a total of ten replicate test specimens. Calculate the arithmetic mean for Basis Weight and report to the nearest 0.01 grams/square meter.

### Material Compositional Analysis

If the information is not otherwise available, the quantitative chemical composition of a test specimen comprising a mixture of fiber types is determined using ISO 1833-1. All measurements are performed in a laboratory maintained at 23° C.±2° C. and 50%±2% relative humidity.

Analysis is performed on test samples taken from rolls or sheets of the raw material, or test samples obtained from a material layer removed from an absorbent article. When excising the material layer from an absorbent article, use care to not impart any contamination or distortion to the layer during the process. The excised layer should be free from residual adhesive. To ensure that all adhesive is removed, soak the layer in a suitable solvent that will dissolve the adhesive without adversely affecting the material itself. One such solvent is THF (tetrahydrofuran, CAS 109-99-9, for general use, available from any convenient source). After the solvent soak, the material layer is allowed to thoroughly air dry in such a way that prevents undue stretching or other deformation of the material. After the material has dried, a test specimen is obtained and tested as per ISO 1833-1 to quantitatively determine its chemical composition.

### Average Fiber Decitex (Dtex) or Denier

Textile webs (e.g., woven, nonwoven, airlaid) are comprised of individual fibers of material. Fibers are characterized in one respect, by their linear mass density, reported in units of denier, or units of decitex. The decitex value is the mass in grams of a fiber present in 10,000 meters of that fiber. The denier value is the mass in grams of a fiber present in 9,000 meters of that fiber. The average decitex or denier value of the fibers within a web of material is often reported by manufacturers as part of a specification. If the average decitex or denier value of the fiber is not otherwise known or available, it can be calculated by measuring the cross-sectional area of the fiber via a suitable microscopy technique such as scanning electron microscopy (SEM), determining the composition of the fiber with suitable techniques such as FT-IR (Fourier Transform Infrared) spectroscopy and/or DSC (Dynamic Scanning calorimetry), and then using a literature value for density of the composition to calculate the mass in grams of the fiber present in 10,000 meters of the fiber (for decitex), or in 9,000 meters of the fiber (for denier).

All testing is performed in a room maintained at a temperature of 23° C.±2.0° C. and a relative humidity of 50%±2% and samples are conditioned under the same environmental conditions for at least 2 hours prior to testing.

If necessary, a representative sample of web material of interest can be excised from an absorbent article. In this case, the web material is removed so as not to stretch, distort, or contaminate the sample.

SEM images are obtained and analyzed as follows to determine the cross-sectional area of a fiber. To analyze the cross section of a sample of web material, a test specimen is prepared as follows. Cut a specimen from the web that is approximately 1.5 cm (height) by 2.5 cm (length) and free from folds or wrinkles. Submerge the specimen in liquid nitrogen and fracture an edge along the specimen's length with a razor blade (VWR Single Edge Industrial Razor blade No. 9, surgical carbon steel). Sputter coat the specimen with gold and then adhere it to an SEM mount using double-sided conductive tape (Cu, 3M available from electron microscopy sciences). The specimen is oriented such that the cross section is as perpendicular as possible to the detector to minimize any oblique distortion in the measured cross sections. An SEM image is obtained at a resolution sufficient to clearly elucidate the cross sections of the fibers present in the specimen. Fiber cross sections may vary in shape, and some fibers may consist of a plurality of individual filaments. Regardless, the area of each of the fiber cross sections is determined (for example, using diameters for round fibers, major and minor axes for elliptical fibers, and image analysis for more complicated shapes). If fiber cross sections indicate inhomogeneous cross-sectional composition, the area of each recognizable component is recorded and dtex contributions are calculated for each component and subsequently summed. For example, if the fiber is bi-component, the cross-sectional area is measured separately for the core and sheath, and dtex contribution from core and sheath are each calculated and summed. If the fiber is hollow, the cross-sectional area excludes the inner portion of the fiber comprised of air, which does not appreciably contribute to fiber dtex. Altogether, at least 100 such measurements of cross-sectional area are made for each fiber type present in the specimen, and the arithmetic mean of the cross-sectional area ax for each are recorded in units of micrometers squared (μm²) to the nearest 0.1 μm².

Fiber composition is determined using common characterization techniques such as FTIR spectroscopy. For more complicated fiber compositions (such as polypropylene core/polyethylene sheath bi-component fibers), a combination of common techniques (e.g., FTIR spectroscopy and DSC) may be required to fully characterize the fiber composition. Repeat this process for each fiber type present in the web material.

The average decitex de value for each fiber type in the web material is calculated as follows:

$${d}_{k} = 10 \; 000 m \times {a}_{k} \times {r}_{k} \times 1 {0}^{- 6}$$

where dx is in units of grams (per calculated 10,000 meter length), ax is in units of μm², and ρₖ is in units of grams per cubic centimeter (g/cm³). Average decitex is reported to the nearest 0.1 g (per calculated 10,000 meter length) along with the fiber type (e.g., PP, PET, cellulose, PP/PET bico). The average denier value for each fiber type in the web material is its decitex dₖ value×0.9.

### Apertures Percent Open Area Measurement Method

Percent open area is measured on images, of an apertured topsheet test specimen, acquired using a flatbed scanner. The scanner is capable of scanning in reflectance mode at a resolution of 2400 dpi and 8 bit grayscale. A suitable scanner is an Epson Perfection V750 Pro from Epson America Inc. (Long Beach, California, USA) or one having substantially similar functionality. The scanner is interfaced with a computer running an image analysis program. A suitable program is ImageJ v. 1.47 (National Institute of Health, USA), or one having substantially similar functionality. The specimen images are distance calibrated against an acquired image of a ruler certified by NIST. To enable maximum contrast, the specimen is backed with an opaque, background sheet of uniformly black color, prior to acquiring the image. All measurement is performed in a conditioned room maintained at about 23° C.±2° C. and about 50±2% relative humidity.

The measurement specimens are prepared as follows.

Obtain the required number of samples of the absorbent article of interest. To obtain a measurement specimen, tape the sample absorbent article about its periphery (i.e., do not tape over regions underlaid by the fluid management layer), wearer-facing side up, in a flat configuration, to a horizontal flat work surface. Any elastic materials included (e.g., in leg cuffs), if present, may be cut to facilitate laying the article out flat. The outer boundary of the region of the apertured topsheet overlying the fluid acquisition layer of the article is identified and marked. Now cut through the topsheet and any adhered underlying layers, about and through this marked outer boundary with a new razor blade or other comparable new, sharp, cutting implement. From this cut out portion, the test specimen of the apertured topsheet is then carefully separated and removed from the underlying layer(s) such that its longitudinal and lateral dimensions are not changed, to avoid distortion of the apertures. If the topsheet is adhered via an adhesive to an underlying layer, before attempting separation apply any solvent suitable for dissolving the adhesive and allowing easy separation of the topsheet from underlying layer(s) without dissolving the polymer material(s) of fibers constituting the topsheet nonwoven web material. (In many examples, tetrahydrofuran (THF) can be a suitable solvent for this purpose. It is not a concern if the solvent dissolves applied surface finish coatings on the fibers, as long as it does not dissolve the polymer(s) constituting the fibers themselves.) Once the cut-out portion of the topsheet constituting the measurement specimen is removed, identify the wearer-facing side thereof. Five replicate measurement specimens obtained from five samples of the absorbent articles of interest, are prepared for measurement. The specimens are conditioned at about 23° C.±2° C. and about 50%±2% relative humidity for 2 hours prior to imaging.

Images are obtained as follows.

The ruler is placed on the scanner bed such that it is oriented parallel to the sides of the scanner glass. An image of the ruler (the calibration image) is acquired in reflectance mode at a resolution of 2400 dpi (approximately 94 pixels per mm) and in 8-bit grayscale. The calibration image is saved as an uncompressed TIFF format file. After obtaining the calibration image, the ruler is removed from the scanner glass and all specimens are scanned under the following scanning conditions.

A measurement specimen is placed onto the center of the scanner bed, lying flat, with the body-facing surface of the specimen facing the scanner's glass surface. The corners and edges of the specimen are secured such that its original longitudinal and lateral dimensions, as on the article prior to removal, are retained. The specimen is oriented such that the long axis and short axis thereof are aligned parallel with and perpendicular to the sides of the scanner's glass surface, respectively. The black background is placed on top of the specimen, the scanner lid is closed, and a scanned image of the entire specimen is acquired with the same settings as used for the calibration image. The specimen image is saved as an uncompressed TIFF format file. The remaining four replicate specimens are scanned and saved in like manner.

The specimen image is analyzed as follows. Open the calibration image file in the image analysis program, and calibrate the image resolution using the imaged ruler to determine the number of pixels per millimeter. Now open the specimen image in the image analysis program, and set the distance scale using the image resolution determined from the calibration image. Now identify a rectangular section (region of interest, or “ROI”) longitudinally and laterally centered on the specimen, having a longitudinal dimension along the longitudinal axis of 60.0 mm and a lateral dimension of 30.0 mm, and visually inspect the images of the apertures present within the ROI. Now using the software tools, manually outline each of the apertures within the ROI (and any partial portions thereof at the edges of the ROI). The appropriate outlines will be drawn along visually discernible inside edges of the concentrations of displaced fibers 503 about the perimeters of the apertures. Stray individual fibers that may have escaped the main structure and/or the concentrations of displaced fibers about the perimeter, and cross into or through the main open area of the aperture (by way of illustrative example, stray individual fibers 504 shown in FIG. 3) are not considered subtractive from the aperture area for purposes herein.) Then use the software to measure the area within each discrete aperture outline (whole and partial) within the ROI and record each to the nearest 0.01 mm², and calculate the sum total thereof. The area of each discrete aperture is defined as the x-y surface area within the visually discernable outline of the open region, created by mechanical penetration of the web and x-y direction displacement of fibers in an aperturing process, that creates the apertures through the web. (For example, refer to FIG. 3 where discrete aperture area 501 and visually discernable boundary 502 are depicted. The dark area of the depicted aperture is an image of black construction paper used as a backing to the specimen of which this particular image was made.) The sum of the areas of all of the apertures within the ROI is recorded as Aperture Arca to the nearest 0.01 mm². Now divide the Aperture Area by the ROI Arca (1,800 mm²), then multiply by 100 and record as Open Area to the nearest 0.1%.

In like manner, repeat the entire procedure for the remaining four replicate specimen images. Calculate the arithmetic mean of Open Area across all five replicate specimens and report as Average Open Area to the nearest 0.1%.

### Acquisition Time and Rewet Measurement Method

This method describes how to measure gush acquisition time, interfacial free fluid amount as well as low and high pressure rewet values for an absorbent article loaded with new Artificial Menstrual Fluid (nAMF), prepared as described herein. A pretreatment step is followed by three introductions of known volumes of nAMF to the absorbent article. The time required for the absorbent article to acquire each of the doses of nAMF is measured using a strikethrough plate and an electronic circuit interval timer. After each liquid dose, Interfacial Free Fluid (IFF) is measured gravimetrically as fluid is transferred from the bottom surface of the strikethrough plate to filter paper. Subsequently, low and high pressure rewet are measured after the last liquid dose. Surface Free Fluid (SFF) is the amount of fluid that remains in the topsheet of the absorbent article. SFF is measured by performing low pressure (0.1 psi) rewet. Immediately after measuring SFF, a higher pressure (0.5 psi) rewet is performed to determine the overall rewet of the absorbent article. All testing is performed in a room maintained at 23° C.±2° C. and 50%±2% relative humidity.

### Equipment and Supplies

### Strikethrough Plate Configuration

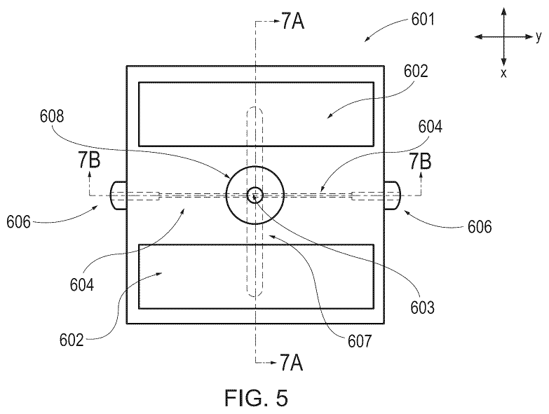

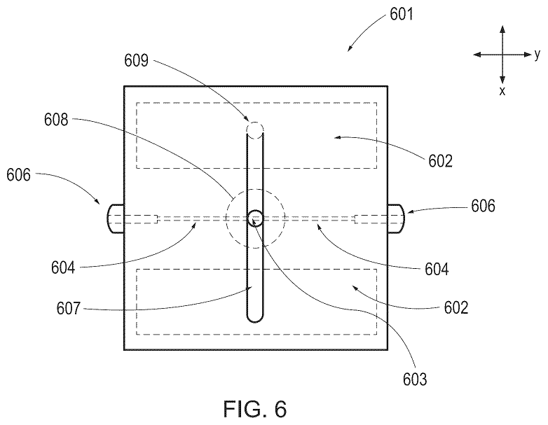

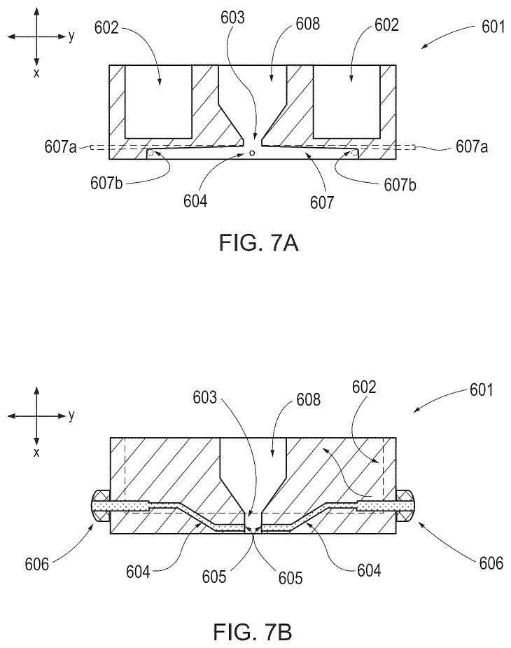

Referring to FIGS. 5, 6, 7A and 7B, the strikethrough plate 601 is made of transparent Plexiglas or equivalent, with an overall dimension of 10.2 cm long (y-direction) by 10.2 cm wide (x-direction) by 3.1 cm tall. (All position and spatial references herein assume an orientation of the strikethrough plate as it would have when resting on a horizontal surface, bottom side down. All references to x-, y- and z-directions in this measurement method description are solely with respect to references to the x-, y- and z-direction arrow indicators appearing in FIGS. 5, 6, 7A and 7B and do not necessarily apply to such references appearing elsewhere in the present specification.) A central, test fluid well 608 with a circular opening and cylindrical wall 25 mm in diameter opens at the top surface of the plate, and extends vertically downwardly (z-direction) from the top surface of the plate to a depth of 15 mm, and then turns radially inwardly to define a conical wall extending vertically downwardly from the top surface by an additional 7.5 mm, while tapering uniformly down to a diameter matching that of the test fluid port 603. The test fluid port 603 is concentric/coaxial with the test fluid well 608 and has a cylindrical wall with a diameter of 6.6 mm, extending further vertically downwardly from the top surface by 5 mm, to a longitudinal fluid channel 607. The longitudinal fluid channel 607 is machined or otherwise formed in the bottom of the plate. The longitudinal fluid channel 607 has a depth from the bottommost surface of the plate defined by vertical side walls that extend upwardly (2-direction) 3.5 mm at the midpoint of the channel (at the test fluid port 603), then slant downwardly at an angle 607a of 0.72° towards each longitudinal end of the channel. The longitudinal fluid channel is open at the bottom surface of the plate, so as to allow fluid to be introduced onto an underlying test specimen, and permitted to flow along the x-y area bounded by the fluid channel 607. The fluid channel 607 is centered under the test fluid port 603 and extends with its length in the in the y-direction that is perpendicular to that of the x-direction paths of the electrodes 604 through the plate. The longitudinal fluid channel 607 has an x-direction width of 5 mm and a y-direction length of 80 mm, upper corners that are rounded with a radius 607b of 1.0 mm about the entire perimeter of the channel. The walls at the opposite distal ends of the longitudinal fluid channel 607 have a cylindrical radius 609 in an x-y plane of 2.5 mm.

Two rectangular cavities 602 (80.5 mm long (x-direction) by 24.5 mm wide (y-direction) by 25 mm deep (z-direction) are symmetrically arranged outboard of the fluid port 603, and centered about a y-direction axis of the plate. These may be loaded with lead shot (or other weighting material) to an extent needed to adjust the total mass of the plate plus the weighting material to provide a pressure of 0.10 psi (7.0 g/cm²) over the total x-y area defined the plate, which is deemed for purposes herein to be 10.2 cm×10.2 cm=104.04 cm², without subtraction of the area defined by the longitudinal fluid channel 607. Electrodes 604 are embedded in the plate 601, each providing an electrical connection between one of the two exterior banana jacks 606 to a position opposite the other, on the inside wall 605 of the longitudinal fluid channel 607. The lowermost portions of the electrodes 604 where their ends are exposed in port 603 is 1.57 mm from the bottommost surface of the plate 601. A circuit interval timer is connected to the jacks 606, so as to monitor the impedance or resistance between the two electrodes 604, and measure the time from introduction of the nAMF into port 603 (establishing an electrical connection between the electrodes and/or substantially decreasing the impedance or resistance therebetween) until the nAMF drains from the port 603 and channel 607 into the test specimen, to a level below the electrodes (breaking the electrical connection between the electrodes and/or substantially increasing the impedance or resistance therebetween). The circuit interval timer has a resolution of 0.01 sec.

### Pretreatment Plate

A pretreatment plate (not shown) is used in combination with a pretreatment weight (not shown) to apply droplets of nAMF to the surface of the test specimen to prime the surface of the specimen prior to the introduction of the full fluid doses specified below. The pretreatment plate is rectangular, made of transparent Plexiglass or equivalent, 14 inch (35.6 cm) long by 8 inch (20.3 cm) wide with a thickness/caliper of about 0.25 inch (6.4 mm). The pretreatment plate is marked with five circular markers, each 5 mm in diameter, placed 1 cm apart (center to center) and centered along the longitudinal axis of the plate. The central marker of the five is centered at the lateral midpoint of the longitudinal axis of the plate. These markers indicate the placement of the nAMF droplets. The markers are located on the underside of the pretreatment plate and can be milled out or simply drawn on with a permanent marker, or equivalent, in any manner such that they are visible through the top surface of the pretreatment plate.

### Pretreatment Weight

The pretreatment weight (not shown) is 10.2 cm×10.2 cm in x- and y-dimensions and consists of a flat, smooth rigid material (e.g., stainless steel). The pretreatment weight has a total mass of 726 g±0.5 g to result in a pressure of 0.10 psi (7.0 g/cm²) across the bottom 104.04 cm² surface area of the pretreatment weight.

### IFF Rubber Pad

When measuring the interfacial fluid amounts, a rubber pad (“IFF rubber pad”) (not shown) with a flat surface is used. The IFF rubber pad is made from high strength neoprene rubber with 40A durometer and a thickness/caliper of ⅛ inch (available from W.W. Grainger, Inc, item #1DUV4, or equivalent) and cut to dimensions of 6 inches (15.2 cm) by 6 inches (15.2 cm).

### Rewet Weight Assembly

For the overall rewet portion of the test, a padded weight assembly (“rewet weight assembly”) (not shown) configured to apply 0.5 psi (35.1 g/cm²) over its 10.2 cm by 10.2 cm (104.04 cm²) surface area is required. The rewet weight assembly is assembled as follows.

Lay a piece of polyethylene film (about 25 microns thick, and about 22.5 cm square in the x-y directions, any convenient source) out flat on a horizontal work surface. A piece of polyurethane foam (25 mm thick, density of 1.0 lb/ft³, IDL 24 psi, available from Concord-Renn Co., Cincinnati, OH, or equivalent) is cut to 10.2 cm by 10.2 cm and then laid in a centered position on top of the film. A piece of transparent Plexiglas (10.2 cm by 10.2 cm and about 6.4 mm thick) is then stacked on top of the polyurethane foam. Next, the polyethylene film is gently pulled taut under the polyurethane foam, and portions of the polyethylene film extending outwardly from under the polyurethane foam are wrapped up and over the polyurethane foam and Plexiglas plate, and secured thereabout with transparent tape. A metal weight of suitable mass is selected and stacked on top of, and fastened to, the Plexiglass plate, such that the total mass of the assembly (rewet weight assembly) is 3.6 kg±0.1 kg.

### Filter Paper

For the IFF, SFF and overall rewet steps, various numbers of layers of filter paper (not shown) are required. The filter paper to be used is to be conditioned at 23° C.±2° C. and 50%±2% relative humidity for at least 2 hours prior to testing. A suitable filter paper has a basis weight of about 88 gsm, a thickness of about 249 microns with an absorption rate of about 5 seconds, and is available from Ahlstrom-Munksjo (Mt. Holly Springs, PA) as grade 632, or equivalent. Each sheet of the filter paper is square, with dimensions of 5 inches by 5 inches (12.7 cm by 12.7 cm).

### Procedure

1) Test samples (examples of absorbent articles of interest) are conditioned at 23° C.±2° C. and 50%±2% relative humidity for at least 2 hours prior to testing.
2) Test samples are removed from their outer packaging and the wrappers are opened to unfold the product, if applicable, using care not to press down or pull on the products while handling. No attempt is made to smooth out wrinkles. Using scissors, cut any adhesive-covering release paper connecting the wings, if present, and lay the sample on a horizontal work surface with the wearer-facing surface facing up (i.e., outward-facing side down).
3) For each sample, determine the dose location as follows. The dose location is the intersection of the midpoints of the longitudinal and lateral axes of the fluid management layer. Once the dose location is identified, mark it with a small dot using a black, fine-tip, permanent marker.
4) For each test of a sample, the test sample is pretreated with nAMF as follows.
   a) Place the pretreatment plate onto a horizontal work surface such that the side with the circular markers is facing down.
   b) Using a single channel, fixed volume pipettor, dispense 50 μL of nAMF onto the topside of the pretreatment plate at each of the five locations overlying each of the five circular markers.
   c) Position the test sample above the pretreatment plate with the wearer-facing surface of the sample down, facing the pretreatment plate, such that the longitudinal axes of the sample and of the pretreatment plate are aligned, and the pre-marked dose location on the test sample is centered over the center droplet of nAMF on the pretreatment plate.
   d) After it is properly positioned over the pretreatment plate, bring the test sample down into contact with the pretreatment plate, and then promptly place the pretreatment weight over/onto the outward-facing (upward-facing) side of the test sample, centering it over the dose location/central droplet of nAMF on the pretreatment plate, and immediately start a stopwatch-type timer set to alarm after 40 seconds. After 40 seconds have elapsed, remove the pretreatment weight from the test sample and remove the test sample from the pretreatment plate. Flip the test sample over so that the wearer-facing side is facing up, place it onto a horizontal work surface, and promptly proceed with the steps that follow.
5) The first acquisition time (ACQ-1) is measured as follows.
   a) Connect the electronic circuit interval timer to the strikethrough plate 601 and zero the timer.
   b) Position the strikethrough plate 601 above the wearer-facing surface of the test sample such that the long (y-direction) axis of the longitudinal fluid channel 607 on the underside of the strikethrough plate 601 is aligned with the longitudinal axis of the test sample, and ensure that the fluid port 603 is centered over the pre-marked dose location on the test sample. The central nAMF droplet applied to the test sample should now be visible through the fluid port 603 at the dose location on the test sample.
   c) After it is properly positioned over the test sample, gently rest the strikethrough plate 601 over/onto the test sample.
   d) Using an adjustable volume pipettor, dispense 2.0 mL of nAMF into the fluid well 608 in the strikethrough plate 601. Dispense the fluid smoothly without splashing, along the conical portion of the wall of the fluid well 608 within a time of 3 seconds or less. When the fluid enters the port 603, electrical connection between the electrodes 604 will be established through the fluid and the circuit interval timer will start timing. When the fluid is acquired by the test sample, electrical connection between the electrodes 604 will be broken and the circuit interval timer will stop timing. Promptly after the circuit interval timer stops timing, start a stopwatch-type timer set to alarm after 2 minutes, leaving the strikethrough plate resting on the test sample during this time. Promptly record the first acquisition time (ACQ-1) displayed on the circuit interval timer, to the nearest 0.1 seconds.
   e) After the 2 minutes have elapsed, measure the first Interfacial Free Fluid (IFF-1) as follows.
      i) Place the IFF rubber pad onto a horizontal work surface. Weigh a first single fresh sheet of the filter paper for this IFF-1 measurement (IFF-1 filter paper sheet), and record the weight as IFF-1ᵢₙᵢₜᵢₐₗ. Place the IFF-1 filter paper sheet over the IFF rubber pad, squared and centered thereover. Promptly lift and move the strikethrough plate 601 from the test sample to the IFF-1 filter paper sheet such that the plate is squared and centered on the filter paper, and immediately start a stopwatch-type timer set to alarm in 8 minutes.
      ii) After 10 seconds have elapsed on the 8 minute timer, remove the strikethrough plate from the IFF-1 filter paper and gently replace it back onto the test sample, exactly as previously positioned.
      iii) Within the next 10 seconds, measure the mass of the IFF-1 filter paper to the nearest 0.0001 g and record as IFF-1_{final}.
6) The second acquisition time (ACQ-2) for the test sample is measured as follows.
   a) After 8 minutes have elapsed per the previously set timer, using an adjustable volume pipettor, dispense a dose of 4.0 mL of nAMF into the fluid well 608 in the strikethrough plate 601. Dispense the fluid smoothly without splashing, along the conical portion of the wall of the fluid well 608 within a time of 3 seconds or less. Promptly after the circuit interval timer stops timing, start a stopwatch-type timer set to alarm after 2 minutes, leaving the strikethrough plate resting on the test sample during this time. Promptly record the second acquisition time (ACQ-2) displayed on the circuit interval timer, to the nearest 0.1 seconds.
   b) After 2 minutes have elapsed, measure the second Interfacial Free Fluid (IFF-2) as follows.
      i) Place the IFF rubber pad onto a horizontal work surface. Weigh a second single fresh sheet of the filter paper for this IFF-2 measurement (IFF-2 filter paper sheet) and record the weight as IFF-2ᵢₙᵢₜᵢₐₗ. Place the IFF-2 filter paper sheet over the IFF rubber pad, squared and centered thereover. Promptly lift and move the strikethrough plate 601 from the test sample to the IFF-2 filter paper sheet such that the plate is squared and centered on the filter paper, and immediately start a stopwatch-type timer set to alarm in 8 minutes.
      ii) After 10 seconds have elapsed on the 8 minute timer, remove the strikethrough plate from the IFF-2 filter paper and gently replace it back onto the test sample, exactly as previously positioned.
      iii) Within the next 10 seconds, measure the mass of the IFF-2 filter paper to the nearest 0.0001 g and record as IFF-2_{final}.
7) The third acquisition time (ACQ-3) is measured as follows.
   a) After 8 minutes have elapsed per the previously set timer, using an adjustable volume pipettor dispense a dose of 2.0 mL of nAMF into the fluid well 608 in the strikethrough plate 601. Dispense the fluid smoothly without splashing, along the conical portion of the wall of the fluid well 608 within a time of 3 seconds or less. Promptly after the circuit interval timer stops timing, start a stopwatch-type timer set to alarm after 2 minutes, leaving the strikethrough plate resting on the test sample during this time. Promptly record the third acquisition time (ACQ-3) displayed on the circuit interval timer, to the nearest 0.1 seconds.
   b) After 2 minutes have elapsed, measure the second Interfacial Free Fluid (IFF-3) as follows.
      i) Place the IFF rubber pad onto a horizontal work surface. Weigh a third single fresh sheet of the filter paper for this IFF-3 measurement (IFF-3 filter paper sheet) and record the weight as IFF-3ᵢₙᵢₜᵢₐₗ. Place the IFF-3 filter paper sheet over the IFF rubber pad, squared and centered thereover. Promptly lift and move the strikethrough plate 601 from the test sample to the IFF-3 filter paper sheet such that the plate is squared and centered on the filter paper, and immediately start a stopwatch-type timer set to alarm in 8 minutes.
      ii) After 10 seconds have elapsed on the 8 minute timer, remove the strikethrough plate from the IFF-3 filter paper sheet and set it on its side so that the bottom side of the plate is not contacting the work surface.
      iii) Within the next 10 seconds, measure the mass of the IFF-3 filter paper sheet to the nearest 0.0001 g and record as IFF-3_{final}.
8) Measure Surface Free Fluid (SFF) as follows. Weigh a first, neat stack of 5 fresh sheets of the filter paper for this SFF measurement (SFF filter paper stack) and record the weight as SFFᵢₙᵢₜᵢₐₗ. After 8 minutes have elapsed per the previously set timer, place the SFF filter paper stack on top of the wearer-facing side of the test sample such that it is centered over the dose location. Now place the strikethrough plate 601 on top of the SFF filter paper stack such that the bottom side of the plate is centered on the filter paper stack, and immediately start a stopwatch-type timer set to alarm in 10 seconds. After 10 seconds have elapsed, remove the strikethrough plate 601 from the filter paper stack and set it aside. Measure the mass of the SFF filter paper stack to the nearest 0.0001 g and record as SFF_{final}. Immediately proceed to the next step.
9) Measure overall rewet as follows. Weigh a second, neat stack of 5 fresh sheets of the filter paper for this REWET measurement (REWET filter paper stack) and record weight as REWETᵢₙᵢₜᵢₐₗ. Place the REWET filter paper stack on top of the wearer-facing side of the test sample such that it is centered over the dose location. Now place the rewet weight assembly on top of the REWET filter paper stack such that the weight is centered on the stack, and immediately start a stopwatch-type timer set to alarm in 30 seconds. After 30 seconds have elapsed, remove the rewet weight assembly and measure the mass of REWET filter paper stack to the nearest 0.0001 g, then record as REWET_{final}.
10) Discard the test sample and thoroughly clean and then dry the strikethrough plate 601 including the fluid well 608, fluid port 603, longitudinal fluid channel 607 and the bottom surface, prior to testing the next sample.
11) Make the following calculations for each of the parameters measured, as follows. Calculate IFF-1 by subtracting IFF-1ᵢₙᵢₜᵢₐₗ from IFF-1_{final}, and record to the nearest 0.0001 g. Calculate IFF-2 by subtracting IFF-2ᵢₙᵢₜᵢₐₗ from IFF-2_{final}, and record to the nearest 0.0001 g. Calculate IFF-3 by subtracting IFF-3ᵢₙᵢₜᵢₐₗ from IFF-3_{final}, and record to the nearest 0.0001 g. Calculate SFF by subtracting SFFᵢₙᵢₜᵢₐₗ from SFF_{final}, and record to the nearest 0.0001 g. Calculate Overall Rewet by subtracting REWETᵢₙᵢₜᵢₐₗ from REWET_{final}, and record to the nearest 0.0001 g.

The entire procedure is repeated for a total of three replicate test samples. The reported value for each of the parameters is the average of the three individually recorded measurements for each Acquisition Time (ACQ-1, ACQ-2 and ACQ-3) to the nearest 0.1 seconds, Interfacial Free Fluid (IFF-1, IFF-2 and IFF-3) to the nearest 0.0001 g, Surface Free Fluid (SFF) to the nearest 0.0001 g and Overall Rewet to the nearest 0.0001 g.

New Artificial Menstrual Fluid (nAMF) Preparation

New Artificial Menstrual Fluid (nAMF) is a mixture of defibrinated sheep blood, a phosphate buffered saline solution and a mucous component. The nAMF is prepared such that it has a viscosity between 7.40 to 9.00 centipoise at 23° C.

Viscosity of the nAMF is measured using a low viscosity rotary viscometer (a suitable instrument is the Brookfield DV2T fitted with a Brookfield UL adapter, available from AMETEK Brookfield, Middleboro, MA, or equivalent). The appropriate size spindle for the viscosity range is selected, and the instrument is operated and calibrated as per the manufacturer. Measurements are taken at 23° C.±1° C. and at 60 rpm. Results are reported to the nearest 0.01 centipoise.

Reagents needed for the nAMF preparation include: defibrinated sheep blood with a packed cell volume of 38% or greater (collected under sterile conditions, available from Cleveland Scientific, Inc., Bath, OH, or suitably comparable source), gastric mucin with a viscosity target of 3-4 centistokes when prepared as a 2% aqueous solution (crude form, sterilized, available from American Laboratories, Inc., Omaha, NE, or suitably comparable source), sodium phosphate dibasic anhydrous (reagent grade), sodium chloride (reagent grade), sodium phosphate monobasic monohydrate (reagent grade), sodium benzoate (reagent grade), benzyl alcohol (reagent grade) and distilled water, each available from VWR International or suitably comparable source.

The phosphate buffered saline solution consists of two individually prepared solutions (Solution A and Solution B). To prepare 1 L of Solution A, add 1.38±0.005 g of sodium phosphate monobasic monohydrate and 8.50±0.005 g of sodium chloride to a 1000 mL volumetric flask and add distilled water to volume. Mix thoroughly. To prepare 1 L of Solution B, add 1.42±0.005 g of sodium phosphate dibasic anhydrous and 8.50±0.005 g of sodium chloride to a 1000 mL volumetric flask and add distilled water to volume. Mix thoroughly. To prepare about 200 mL of phosphate buffered saline solution, add 49.50 g±0.10 g of Solution A and 157.50 g±0.10 g of Solution B to a sufficiently size bottle that has a lid with a good seal. Then add 1.0 g of sodium benzoate and 1.60 g of benzyl alcohol to the bottle along with a stir bar and set aside.

The mucous component of the nAMF is a mixture of the phosphate buffered saline solution and gastric mucin. The amount of gastric mucin added to the mucous component directly affects the final viscosity of the prepared nAMF. To determine the amount of gastric mucin needed to achieve nAMF within the target viscosity range (7.4-9.0 centipoise at 23° C. and 60 rpm), prepare 3 batches of nAMF with varying amounts of gastric mucin in the mucous component, and then interpolate the exact amount needed from a concentration versus viscosity curve with a least squares linear fit through the three points. A successful range of gastric mucin is usually between 13 to 15 grams per 400 mL batch of nAMF, although this can vary significantly based upon the supplier, age, and lot (production batch) of mucin.

To prepare about 200 mL of the mucous component, add the pre-determined amount of gastric mucin to the bottle containing the previously prepared phosphate buffered solution and then apply the lid. Place the bottle on a wrist-action shaker for 5 minutes at the highest speed. After 5 minutes, remove the flask of mucous component from the wrist-action shaker and place onto a magnetic stir plate. Stir for at least 2 hours until there are no lumps of mucin present, then remove the stir bar from the flask. Using a homogenizer, blend the mucous component for 5 minutes at 10,000 rpm. A suitable homogenizer is the T18 Ultra-Turrax fitted with a S18N-19G dispersing tool (19 mm stator diameter, 12.7 mm rotor diameter, 0.4 mm gap between rotor and stator), both available from IKA Works, Inc, Wilmington, NC, or suitably comparable source. After the final mixing step, measure and record the viscosity of the mucous component to the nearest 0.01 centipoise at 23° C.±1° C. and at 20 rpm using the viscometer with the UL adapter. Ensure that the viscosity of the prepared mucous component is within the target range of 9.0-11.0 centipoise.

The nAMF is a 50:50 mixture of the mucous component and sheep blood. Ensure the temperature of the sheep blood and mucous component are 23° C.±1° C. To prepare about 400 mL of nAMF, add 200 g of the mucous component to a glass bottle with at least 500 mL capacity. Now add 200 g of sheep blood to the bottle along with a stir bar. Mix on a magnetic stir plate until thoroughly combined. Ensure the viscosity of the prepared nAMF is within the target range of 7.4-9.0 centipoise when measured at 23° C.±1° C. and 60 rpm using the viscometer with the UL adapter. If the viscosity is too high, it can be adjusted by adding the previously prepared phosphate buffered saline solution in 0.5 g increments followed by stirring for 2 minutes and then re-checking the viscosity until the target range is reached.

The qualified nAMF should be refrigerated at 4° C. unless intended for immediate use. nAMF may be stored in an air-tight container at 4° C. for up to 48 hours after preparation. Prior to testing, the nAMF must be brought to 23° C.±1° C. Any unused portion is discarded after testing is complete.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “approximately 40 mm.”

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article comprising:
a liquid permeable topsheet having an inner surface oriented toward an interior of the absorbent article and an outer surface oriented toward the skin of a wearer when the absorbent article is being worn, said topsheet comprising individual nonwoven fibers;
a backsheet;
an absorbent core disposed between the topsheet and the backsheet; and
a topsheet lotion composition;
wherein the topsheet lotion composition comprises discrete drops on individual fibers of the topsheet;
wherein the discrete drops are on both the outer surface and between the outer surface and inner surface of the topsheet;
wherein each drop is at most about 100 microns; and
wherein the lotion composition has a viscosity from about 3.0 cP to about 5.0 cP at 80° C.

2. The absorbent article of claim 1, wherein the topsheet has an outer surface area and the lotion composition is on from about 30% to about 90% of the topsheet outer surface area.

3. The absorbent article of claim 1, wherein from about 10% to about 40% of the lotion composition drops are between the outer surface and the inner surface of the topsheet.

4. The absorbent article of claim 1, wherein the lotion composition comprises
a rheology structurant;
a surfactant; and
a carrier.

5. The absorbent article of claim 4, wherein the lotion composition comprises from about 8 wt % to about 90 wt % of the rheology structurant.

6. The absorbent article of claim 4, wherein the ratio of the wt % of carrier to wt % of rheology structurant is from about 9:1 to about 7.5:2.5.

7. The absorbent article of claim 4, wherein the lotion composition comprises about 10 wt % to about 25 wt % of a rheology structurant selected from a fatty alcohol having from about 12 to about 24 carbon atoms and/or a fatty monoester having from about 40 to about 44 carbon atoms.

8. The absorbent article of claim 4, wherein the lotion composition comprises at most about 1 wt % of a surfactant that is a polypropylene glycol material.

9. The absorbent article of claim 8, wherein the polypropylene glycol is selected from POE-4 monolaurate, POE-4 dilaurate, POE-8 dioleate, Oleth-2, Oleth-3, PPG-15 stearyl ether, and PPG-3 myristyl ether, and combinations thereof.

10. The absorbent article of claim 4, wherein the lotion composition comprises from about 5% to 99% by weight of a carrier that comprises plant-based liquid triglycerides.

11. The absorbent article of claim 4, wherein the carrier comprises a plant-based liquid triglyceride; wherein the liquid triglyceride is a C10-C18 triglyceride.

12. The absorbent article of claim 4, wherein the carrier is caprylic/capric triglyceride.

13. The absorbent article of claim 4, wherein the carrier comprises caprylic/capric triglyceride that is a mixture of C8 and C10 at a ratio from 70:30 to 50:50.

14. The absorbent article of claim 4, wherein the carrier comprises caprylic/capric triglyceride, wherein the ratio of caprylic triglyceride to capric triglyceride is about 60 to 40.

15. The absorbent article of claim 1, wherein the topsheet comprises a nonwoven web of carded staple length fibers having an average staple length of 38 mm and an average denier of 1.5 to 4.

16. The absorbent article of claim 14, wherein the fibers are bicomponent with a concentric sheath-core configuration; wherein the core component is PET and the sheath component is low density polyethylene (LDPE); and wherein the weight ratio of the components is about 1:1.

17. The absorbent article of claim 15, wherein the topsheet comprises a plurality of randomly-distributed fiber-to-fiber bonds created via air-through heat bonding of carded fibers.

18. The absorbent article of claim 1, wherein the topsheet lotion is semi-solid or solid at room temperature and liquid above 50° C.

19. The absorbent article of claim 1, wherein the absorbent article has a Total Rewet+SFF value from about 20 to about 300 mg, as measured according to the SFF and Rewet Method.

20. The absorbent article of claim 1, wherein the lotion composition comprises an elastic modulus of from about 5 to about 25,000 dyne/cm².
